(19) United States
(12) Patent Application Publication
(10) Pub. No.: US 2021/0270843 A1
(43) Pub. Date: Sep. 2, 2021

(54) Immune Monitoring of Neuro-Inflammatory Amyotrophic Lateral Sclerosis (ALS)

(71) Applicant: RAPA THERAPEUTICS, LLC, Rockville, MD (US)

(72) Inventor: Daniel Harding Fowler, Bethesda, MD (US)

(21) Appl. No.: 17/320,996

(22) Filed: May 14, 2021

Related U.S. Application Data
(63) Continuation of application No. PCT/US2019/061803, filed on Nov. 15, 2019.
(60) Provisional application No. 62/768,187, filed on Nov. 16, 2018.

(51) Int. Cl. G01N 33/68; C12N 5/0783; A61K 38/17; A61K 39/395; A61K 45/06; A61K 35/17; G01N 33/569

(52) U.S. Cl. CPC G01N 33/6893; C12N 5/0636; G01N 33/6863; A61K 38/1793; A61K 39/3955; A61K 45/06; G01N 2800/52; G01N 33/56966; C12N 2500/84; C12N 2501/999; G01N 2800/24; C12N 2501/52; A61K 35/17

(57) ABSTRACT

The present disclosure provides methods for monitoring inflammation in ALS and other related diseases. Therapeutic interventions based on the results of monitoring methods are also provided.

### CROSS-REFERENCE TO RELATED APPLICATIONS

The present application claims priority to U.S. Provisional Application No. 62/768,187, filed Nov. 16, 2018, the entirety of which is incorporated herein by reference.

### BACKGROUND

Amyotrophic lateral sclerosis (ALS) is a primary neurodegenerative disease involving the cerebral cortex, brainstem, and spinal cord that results in progressive disability and typically death due to respiratory failure. ALS is a familial disease in 10% of patients due to various genetic events; the remainder of patients have sporadic ALS, where the etiology is not known but may involve environmental factors. The most recent registry data (2013) indicates that the prevalence of ALS in the United States was approximately 16,000 cases; these data also indicate that ALS disproportionately affects whites, males, and individuals in the 60 to 69 age group. ALS is a heterogeneous disease with various clinical presentations and rates of progression. Although the average survival of ALS patient is between two to four years from diagnosis, survival can be as short as months or over a decade.

It is difficult to estimate prognosis in ALS patients because disease scoring systems such as the patient-reported ALSFRS-R score (ALS Functional Rating Score, Revised) do not account for the linear and non-linear aspects of disease progression. This difficulty in estimating the rate of disease progression represents a limitation for clinical trials in ALS and indicates that potential disease biomarkers, including the immunologic monitoring of the present disclosure, should be emphasized as a component of protocol therapy. The clinical onset of ALS is insidious, with most patients presenting with upper or lower limb weakness or speaking or swallowing difficulty (bulbar-onset). ALS remains a diagnosis of exclusion, as there are no definitive blood, spinal fluid, or radiologic exams; as a result, ALS is typically a diagnosis of exclusion after other diseases have been ruled-out. This process of ruling out other diseases can typically take up to one year and thereby delays therapeutic attempts and clinical trial accrual; this delay in referral likely has consequences because, at the time of eventual ALS diagnosis, up to 50% of motor neurons may no longer be functional. Given this situation, it is typically recommended to accrue ALS patients to investigational trials at a relatively early point after diagnosis. Towards this aim, the approach to patient monitoring that we detail will be beneficial in terms of early diagnosis and early intervention efforts.

Riluzole (Rilutek®), which was the first drug approved for ALS therapy in 1995, is only mildly efficacious in reducing the morbidity and mortality of ALS. Despite significant clinical research whereby more than 60 molecules have been investigated for ALS therapy, there have been only two additional molecules that have shown modest clinical success, namely the anti-oxidant edaravone and the tyrosine kinase inhibitor masitinib. Edaravone (Radicava®), which was recently FDA-approved for therapy of ALS, provides minimal clinical benefit, is expensive, and requires a 2-weeks on, 2 weeks-off daily continuous i.v. infusion therapy; masitinib is not FDA-approved. As such, given the current state of very limited therapeutic options, there is a great need to evaluate novel strategies for the therapy of ALS; in the context of developing such novel therapeutics, it will be essential to accurately and comprehensively monitor the ALS patient's inflammatory state.

ALS is a primary neurodegenerative disease, with neuro-inflammation acting as a secondary, propagating factor. Evidence for this conclusion is derived in part from the observation that a functional abnormality in the TAR DNA-binding protein 43 (TDP-43) occurs in the vast majority of familial and sporadic ALS patients. TDP-43, which in the healthy state is restricted to the nucleus, is an RNA and DNA binding protein that is susceptible to aggregation, thereby accounting in part for the cytoplasmic inclusion bodies seen in the neurons of ALS patients. The precise mechanisms that result in alteration of the TDP-43 pathway remain to be fully elucidated, but appear to involve various cellular stress events or amplification of genomic elements (retrotransposable elements, RTE) that replicate themselves via RNA intermediates. Ultimately, such events lead to a multi-faceted programmed cell death in neurons, including programmed necrosis. Of note, the necrotic cell death pattern that occurs in ALS patients has been shown to be particularly immunogenic relative to the more orderly apoptotic cell death; indeed, TNF-α, which is a known molecular mediator of motor neuron death in ALS, can produce the necrotic form of cell death. Necrotic cell death can lead to the release of self-antigens that can then be presented to the adaptive immune system for the induction of autoimmunity; in addition, because protein aggregates themselves may be immunogenic, it is possible that protein aggregates that occur in ALS patients (including but not limited to TDP-43; SOD-1; p62) might be targets of an autoimmune response that emanates after neurodegeneration. Indeed, it has recently been shown that monocytes from ALS patients develop an inflammatory phenotype when pulsed with exosomes containing TDP-43.

In response to primary neurodegeneration, there is broad evidence that the innate inflammasome and the adaptive peripheral immune system combine to elicit further ALS disease progression. In the superoxide dismutase-1 (SOD1) transgenic mouse model of ALS, CD3⁺ T cell infiltration of the spinal cord and microglial cell activation were recognized as pro-inflammatory factors that contributed to disease progression. Furthermore, transfer of wild-type microglial cells with reduced inflammatory propensity relative to host microglial cells in the PU.1 knockout mouse model of ALS reduced neurodegeneration and improved survival. In addition, a protective role for CD4⁺ T cells was described for the first time in the SOD1 murine model of ALS, thereby indicated the double-edged sword nature of the peripheral immune T cell pool in ALS (acting as either propagating or protective factor). In subsequent studies, the phenotype of the protective CD4⁺ T cell subset in the SOD1 murine model of ALS was characterized as a regulatory T (T_{REG}) cell population that reduced inflammation through a mechanism mediated in part through the counter-regulatory Th2-type cytokines IL-4 and IL-β.

In ALS patients, direct evidence for the deleterious role of the peripheral adaptive immune system T cells can be ascertained by the demonstration that T cells infiltrating the spinal cord express an oligoclonal T cell receptor (TCR) repertoire. Furthermore, professional antigen-presenting-cells (dendritic cells) emanating from the peripheral immune system can be isolated in ALS patient spinal cord tissue in close association with inflammatory periphery-derived monocytes and resident CNS microglial cells. Additionally, in ALS patients, purified monocytes express a pro-inflammatory RNA expression profile, including an increase in the innate inflammatory molecule IL-1-β, which can then drive the IL-23 pathway that promotes CD4⁺ T-helper-1 (Th1), CD8⁺T-cytotoxic-1 (Tc1), and CD4⁺ Th17-mediated neurodegenerative immunity.

This biology is consistent with an abundance of data in neuro-inflammation research indicating that: microglial cells are a key cellular constituent in the brain that drives neurodegeneration; and microglial cells and CNS-infiltrating peripheral CD4⁺ T cells interact and influence disease pathogenesis. Consistent with the murine modeling results, patients with a peripheral immune system enriched for FoxP3⁺ T_{REG} cells and Th2-type T cells had a reduced progression rate of ALS relative to patients with primarily a pro-inflammatory Th1-type immune profile. Furthermore, it was recently found that ALS patient T_{REG} cells are dysfunctional, with such dysfunction correlating with disease progression rate and severity. Combined, these results have provided a rationale for investigation of adoptive T cell therapy using purified and ex vivo expanded natural (n) T_{REG} cells for therapy of ALS. A different subset of T_{REG} cells, the induced (i) form known as iT_{REGS} has been developed. Such iT_{REG} cells are not derived from the thymus as in the nT_{REG} cell population; rather, the iT_{REGS} are a population that is converted from otherwise pathogenic post-thymic T cell subsets such as Th1 cells. Although both nT_{REGS} and iT_{REGS} play important and non-redundant roles in the dampening of inflammatory responses, development of an iT_{REG} therapy is relatively advantageous in terms of regulatory T cell potency and ease of manufacturing.

In sum, these data provide evidence that the primary neurodegenerative process in ALS gives rise to a secondary inflammatory response that on the one hand can drive disease progression yet on the other hand can point to therapeutic interventions at multiple steps, including: control of inflammasome activation, depletion and suppression of Th1/Tc1-type subsets, and promotion of T_{REG}-type subsets. Given this information, there exists great interest in evaluating immune modulation therapies in ALS patients. These ALS therapy efforts will be particularly effective if used in combination with the immune monitoring techniques that we describe here.

### SUMMARY

The present disclosure is directed to methods for determining the inflammatory state of a subject suffering from amyotrophic lateral sclerosis (ALS).

In an embodiment, a method comprises culturing peripheral blood mononuclear cells (PBMCs) comprising CD14⁺ monocytes and CD3⁺ T cells from a subject suffering from ALS in a culture medium supplemented with human serum and CGS21680 or a salt thereof for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state.

In another embodiment, a method comprises culturing peripheral blood mononuclear cells (PBMCs) comprising CD14⁺ monocytes and CD3⁺ T cells from a subject suffering from ALS in a culture medium supplemented with human serum and CD40 ligand (CD40L) for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state.

In any of the foregoing embodiments, the method may further comprise prior to culturing said PBMCs: isolating said CD14⁺ monocytes and CD3⁺ T cells from a sample comprising said PBMCs from said subject.

In any of the foregoing embodiments, the method may further comprise prior to isolating said CD14⁺ monocytes and CD3⁺ T cells: harvesting a sample comprising PBMCs from said subject.

In any of the foregoing embodiments, the method may further comprise, if said subject is in an inflammatory state and said at least one cytokine is TNF-α: administering an anti-TNF-α therapy. In some embodiments, a therapy that preferentially neutralizes the cell-free, soluble form of TNF-α while sparing the membrane-bound form of TNF-α, including but not limited to the recombinant receptor for TNF-α (etancercept; Enbrel®) or the anti-TNF-α monoclonal antibody adalimumab can be administered.

In any of the foregoing embodiments, the method may further comprise, if said subject is in an inflammatory state and said at least one cytokine is IL-1β: administering an anti-IL-1β therapy, potentially in combination with an anti-TNF-α therapy such as, by way of example but not limitation, etanercept, which will indirectly reduce IL-1β levels.

In any of the foregoing embodiments, the method may further comprise, if said subject is in an inflammatory state and said at least one cytokine is IL-6: administering an anti-IL-6 therapy, potentially in combination with an anti-TNF-α therapy such as, by way of example but not limitation, etanercept, which will indirectly reduce IL-6 levels.

In any of the foregoing embodiments, prior to collection of T cells by apheresis for input into T_{REG} manufacturing, the subject may be treated with one of a select group of anti-TNF-α reagents that preferentially alters the TCR repertoire for TNFR2-expressing input T cells that are shifted towards a T_{REG} phenotype. Alternatively, the said selective anti-TNF-α therapy can be followed by any other intervention designed to further promote T_{REG} cells in vivo, including but not limited to low-dose IL-2 therapy or rapamycin therapy.

In any of the foregoing embodiments, the subject can be considered in an inflammatory state and therefore in need of immune modulation therapy if the RNA-based T cell receptor repertoire analysis and associated laboratory investigations indicate that the subject expresses an increase in T cell clonality, particularly when found in association with an increased biologic drive towards T cell clonality.

In any of the foregoing embodiments, the subject can be considered in an inflammatory state if the subject expresses an increased biologic drive towards an increase in T cell clonality (with or without an actual increase in T cell clonality), as evidenced by presence of markers including but not limited to: an increase in the number of TCR sequences that are due to alternative splicing of RNA; evidence of an increase in T cell expression of master regulators of alternative splicing, including but not limited to hnRNPLL; or evidence of expression of down-stream molecules indicative of an increase in alternative splicing master regulators.

In any of the foregoing embodiments, the method may further comprise, if said subject is in an inflammatory state: subjecting said subject to an immune depletion regimen followed by adoptive transfer of natural regulatory T cells (nT_{REGS}), manufactured regulatory T cells (iT_{REGS}), or a combination thereof.

In any of the foregoing embodiments, the method may further comprise, if such subject is in an inflammatory state: first subjecting such a subject to a therapy such as etanercept to reduce T cell clonality and to modulate the TCR repertoire towards a T_{REG} phenotype prior to the collection by apheresis of T cells utilized in the manufacture of nT_{REG} cells or iT_{REG} cells.

### BRIEF DESCRIPTION OF THE DRAWINGS

FIG. 1A depicts transcription factor flow cytometry of peripheral blood mononuclear cells from a normal control.

FIG. 1B depicts transcription factor flow cytometry of peripheral blood mononuclear cells from an ALS patient.

FIG. 2A depicts CD86 expression in the CD16⁺ monocyte subset of a normal control detected by flow cytometry.

FIG. 2B depicts CD86 expression in the CD16⁺ monocyte subset of an ALS patient detected by flow cytometry.

FIG. 3 depicts ALS patient and normal control autonomous cytokine secretion.

FIG. 4 depicts an example of ALS patient autonomous cytokine secretion that is modulated by anti-TNF therapy.

FIG. 5 depicts an example of CD40L-driven cytokine secretion in ALS patients.

FIG. 6 depicts evaluation of autonomous cytokine secretion in a sibling of a subject who was a carrier for the C9orf72 mutation.

FIG. 7 depicts adenosine receptor-modulated cytokine secretion in an ALS patient and normal control.

FIG. 8 depicts cytokine secretion from an ALS patient and normal control following PD1 pathway blockade.

FIG. 9 depicts cytokine secretion from an ALS patient and normal control following CD200 pathway blockade.

FIG. 10A depicts flow cytometry distribution of CD4⁺ and CD8⁺ T cell subsets and expression of the Th1-type transcription factor TBET within the CD8⁺ T cell subset in a normal control.

FIG. 10B depicts flow cytometry distribution of CD4⁺ and CD8⁺ T cell subsets and expression of the Th1-type transcription factor TBET within the CD8⁺ T cell subset in an ALS patient.

FIGS. 11A-11C depict use of RNA-based T cell receptor sequencing (clonality analysis) to differentiate subjects with ALS from normal controls and to monitor ALS subject inflammatory disease response to a therapeutic intervention.

FIGS. 12A-12B depict use of RNA-based T cell receptor sequencing to identify the widespread changes in T cell receptor up- and down-regulation in ALS patients due to anti-TNF-αtherapy (etanercept).

FIG. 13 depicts IRF3 and IRF7 expression in peripheral blood mononuclear cells of a normal control and an ALS patient.

FIG. 14 depicts cyclic AMP measurement as a novel approach to monitor the inflammatory state in patients with neurodegenerative diseases such as ALS. Peripheral blood mononuclear cells were isolated from a patient with ALS and a normal control. The cells were incubated (1×10⁶ cells per condition) in the presence of the adenosine A_{2A} receptor agonist CGS21680 (concentration, 0.1 μM) and a cell lysate was obtained at the indicated time points to harvest cyclic AMP. Cyclic AMP was then measured using a commercially available, competitive ELISA test.

### DETAILED DESCRIPTION

### Definitions

As used herein, the singular forms “a”, “an” and “the” include plural referents unless the context clearly dictates otherwise.

The use of the term “or” in the claims and the present disclosure is used to mean “and/or” unless explicitly indicated to refer to alternatives only or the alternatives are mutually exclusive.

Use of the term “about”, when used with a numerical value, is intended to include +/−10%. For example, if a number of amino acids is identified as about 200, this would include 180 to 220 (plus or minus 10%).

The terms “patient,” “individual,” and “subject” are used interchangeably herein, and refer to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Sample” is used herein in its broadest sense. A sample comprising cells, polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Treatment” is an intervention performed with the intention of preventing the development or altering the pathology or symptoms of a disorder. Accordingly, “treatment” can refer to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

“Immune cells” as used herein, is meant to include any cells of the immune system that may be assayed, including, but not limited to, B lymphocytes, also called B cells, T lymphocytes, also called T cells, natural killer (NK) cells, natural killer T (NKT) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“T cells” are a subset of lymphocytes originating in the thymus and having heterodimeric receptors associated with proteins of the CD3 complex (e.g., a rearranged T cell receptor, the heterodimeric protein on the T cell surfaces responsible for antigen/MHC specificity of the cells). T cell responses may be detected by assays for their effects on other cells (e.g., target cell killing, activation of other immune cells, such as B-cells) or for the cytokines they produce.

As used herein, the term “immune depletion regimen” can be any regimen that depletes immune cells, including but not limited to T cells, Th1 cells, and/or Tc1 cells, unless otherwise noted.

As used herein “manufactured induced regulatory T cells (iT_{REGS})” refers to any manufactured T_{REG} cell, e.g. T_{REG} cells or T_{REG} hybrid cells obtained by ex vivo culture that are manufactured from a T cell substrate that is not enriched for thymic-derived CD4⁺ natural regulatory T cells. In contrast, the term “nT_{REG}” as used herein refers to naturally-occurring T_{REG} cells derived from the thymus or T cells manufactured from the nT_{REG} cell population.

As used herein, the term “therapeutically effective dose” or “therapeutically effective amount” is meant an amount of a compound of the present invention effective to yield the desired therapeutic response. By way of example but not limitation, a dose effective to delay the growth of or to cause the cancer to shrink or prevent metastasis can be a “therapeutically effective dose.” The specific therapeutically effective dose will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

The methods of the present disclosure can characterize the inflammatory state of ALS patients by approaches that have not been specifically evaluated in the literature. We reason that such in-depth immune characterization of ALS patients will be beneficial in terms of: (1) improving an understanding of disease pathogenesis, thereby providing insights into new therapeutic approaches; (2) allowing early diagnosis, which will improve patient outcome by allowing early interventions that will be more effective at reducing the inflammatory state and will preserve neuronal function; and (3) providing bio-markers for therapeutic interventions.

The present disclosure provides a comprehensive method, and specific assays, for monitoring the inflammatory state of ALS patients that can include the following: (1) use of flow cytometry to evaluate key issues in the field such as co-expression of multiple transcription factors or a suppressor transcription factor (FoxP3) in combination with inflammatory cytokines (IL-2, IFN-γ); (2) use of autonomous cytokine secretion to detect an inflammatory state; (3) use of several methods to enhance cytokine secretion, including T cell checkpoint inhibition, monocyte checkpoint inhibition, CD40 ligand exposure, and adenosine receptor modulation; (4) measurement of T cell cyclic AMP response to adenosine receptor modulation as a readout for compensatory blunting of the host response to neuro-inflammation; (5) use of Western Blot to characterize the post-CD40 signaling cascade and the NLRP3 signaling cascade; (6) use of high throughput RNA sequencing to characterize the TCR repertoire pre- and post-therapy for quantification of overall TCR clonality, contribution of alternative splicing to the TCR repertoire, and identification of both clonal expansion and clonal deletion events; (7) use of peptide pool technology to identify ALS patient sensitization to protein aggregates associated with disease progression including but not limited to SOD-1, TDP-43, and p62; and (8) detection of T cell reactivity to necrotic motor neuron material. The methods of the present disclosure can be performed independently or as part of a panel, for example, including two or more methods.

These methods for monitoring the inflammatory state in ALS will have multiple beneficial effects, including: (1) assessment of disease progression risk for more appropriate allocation of therapeutic options; (2) assessment of disease development risk in carriers of genes associated with ALS predisposition; and (3) the monitoring of disease during and after therapeutic interventions.

In an embodiment, the method comprises culturing peripheral blood mononuclear cells (PBMCs) comprising CD14+ monocytes and CD3+ T cells from a subject suffering from ALS in a culture medium supplemented with human serum and CGS21680 or a salt thereof for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state. The chemical structure of CGS21680 is shown below:

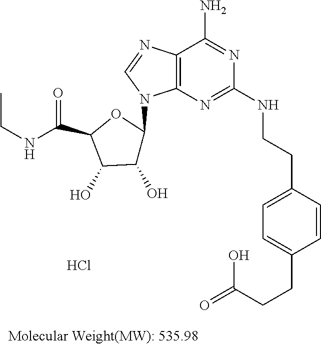 [SMILES: CCNC(=O)[C@H]1O[C@@H](n2cnc3c(N)nc(NCCc4ccc(CCC(=O)O)cc4)nc32)[C@H](O)[C@@H]1O ; Cl]

In another embodiment, the method comprises culturing peripheral blood mononuclear cells (PBMCs) comprising CD14⁺ monocytes and CD3⁺ T cells from a subject suffering from ALS in a culture medium supplemented with human serum for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state.

In another embodiment, the method comprises culturing peripheral blood mononuclear cells (PBMCs) comprising CD14⁺ monocytes and CD3⁺ T cells from a subject suffering from ALS in a culture medium supplemented with human serum and CD40 ligand (CD40L) for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state.

In another embodiment, the method comprises culturing peripheral blood mononuclear cells (PBMCs) comprising CD14⁺ monocytes and CD3⁺ T cells from a subject suffering from ALS in a culture medium supplemented with human serum and an anti-PDL1 or anti-PD1 antibody or fragment thereof for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state.

In another embodiment, the method comprises culturing peripheral blood mononuclear cells (PBMCs) comprising CD14⁺ monocytes and CD3⁺ T cells from a subject suffering from ALS in a culture medium supplemented with human serum and an anti-CD200L or anti-CD200 antibody or fragment thereof for a period of time to yield a conditioned culture medium comprising a conditioned supernatant; collecting said conditioned supernatant; measuring a concentration of at least one cytokine selected from TNF-α, IL-1β and IL-6 in said conditioned supernatant; comparing said concentration of said at least one cytokine to a concentration of said at least one cytokine in a control sample or a standard value, wherein an increase in the concentration of the at least one cytokine in the conditioned supernatant relative to the concentration of the at least one cytokine in the control sample or a standard value is indicative that said subject is in an inflammatory state.

In any of the foregoing embodiments, where the culture is supplemented with an additional component in addition to human serum, a control sample can be from a culture performed under the same conditions without the additional component. For example, a control sample for the immediately foregoing embodiment where an anti-CD200L or anti-CD200 antibody or fragment thereof is added, would be from a culture of PMBCs comprising CD14⁺ monocytes and CD3⁺ T cells from the subject cultured under the same conditions as the sample to be tested.

In any of the foregoing embodiments, said culture medium can include X-Vivo 20 media, however, any suitable media for culturing T cells and/or monocytes ex vivo can be used. In some embodiments, said culture medium is supplemented with 5% human serum. By way of example but not limitation, culture media can be supplemented with at least 1%, 2%, 3%, 4%, 5%, 6,%, 7%, 8%, 9%, 10%, 11%, 12,% 13%, 14%, 15%, 16%, 17%, 18%, 19%, 20% human serum and any range comprising values therebetween. In some embodiments, said culture medium contains, by way of example but not limitation, 0.1 to 10 μg/mL, 0.1 to 5 μg/mL, 0.1 to 1 μg/mL, 0.1 to 0.5 μg/mL, 1 to 10 μg/mL, 1 to 5 μg/mL, 5 to 10 μg/mL, or 0.1 μg/mL, 0.5 μg/mL, 1 μg/mL, 5 μg/mL, or 10 μg/mL CD40L. In some embodiments, said CD40L is recombinant, human CD40L. In some embodiments, said culture medium contains, by way of example, but not limitation, 0.001 to 10 μg/mL, 0.01 to 10 μg/mL, 0.1 to 10 μg/mL, 1 to 10 μg/mL, 0.001 to 1 μg/mL, 0.01 to 1 μg/mL, 0.1 to 1 μg/mL of the anti-PDL1 or anti-PD1 antibody or fragment thereof. In some embodiments, said culture medium contains, by way of example, but not limitation, 0.01 to 10 μg/mL, 0.1 to 10 μg/mL, 1 to 10 μg/mL, 0.01 to 1 μg/mL, or 0.1 to 1 μg/mL of the anti-CD200L or anti-CD200 antibody or fragment thereof.

In some embodiments, said culture medium contains, by way of example but not limitation, 0.01 to 10 μM, 0.01 to 5 μM, 0.01 to 1 μM, 0.01 to 0.05 μM, 0.05 to 10 μM 0.05 to 5 μM, 0.05 to 1 μM, 0.05 to 0.1 μM, 0.1 to 5 μM, 0.1 to 1 μM, 0.1 to 0.5 μM, 0.5 to 5 μM, 0.5 to 1 μM, 1 to 5 μM, or 10 μM, 5 μM, 1 μM, 0.5 μM, 0.1 μM, 0.05 μM, or 0.01 μM of CGS21680 of the salt thereof. In some embodiments, the modulator of adenosine receptor signaling can consist of the natural ligand or any pharmaceutically-produced ligand, by way of example but not limitation as those described in Jacobson KA, Müller CE. Medicinal Chemistry of Adenosine, P2Y and P2X Receptors. Neuropharmacology. 2016; 104:31-49. Specifically, molecules that can be used include but are not limited to: the natural ligand adenosine; inosine; adenosine 5′N-ethyluraonamide; regadenoson (CVT-3146); istradefylline (KW-6002); trabodenoson (INO-8875); capadenoson (BAY68-4986); piclidenoson; ABT-702; TRR469; and MRS1191.

In some embodiments, said period of time is, by way of example but not limitation, from 18 to 48 hours, 18 to 42 hours, 18 to 36 hours, 18 to 30 hours, 18 to 24 hours, 24 to 48 hours, 30 to 48 hours, 36 to 48 hours, 42 to 48 hours, 36 to 42 hours, 30 to 42 hours, 24 to 42 hours, 24 to 36 hours, 24 to 30 hours, 30 to 36 hours, or 48 hours, 42 hours, 36 hours, 30 hours, 24 hours, or 18 hours.

In some embodiments, said increase in the concentration of the at least one cytokine in the conditioned supernatant relative to the concentration of the at least one cytokine in the control sample is at least a three-fold increase. By way of example but not limitation, at least a three-fold increase in secretion of any one of the following cytokines relative to control stimulation condition, IFN-gamma, GM-CSF, or TNF-alpha.

In some embodiments, cells to be tested can be inoculated into culture at a standard culture density. By way of example but not limitation, the culture density at inoculation can be about 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, 10×10⁶ cells/mL, 15×10⁶ cells/mL, 20×10⁶ cells/mL, 30×10⁶ cells/mL, or any value therebetween or any range thereof.

In some embodiments, said standard value for cytokine secretion is 10 pg/mL/1×10⁶ cells/24 hours or less. By way of example but not limitation, said standard value can be about 1, 2, 3, 4, 5, 6, 7, 8, 9 or 10 pg/mL/1×10⁶ cells/24 hours.

In some embodiments where the culture is supplemented, said supplementation can be before inoculation of cells into the culture medium or by adding said components at a later time.

In certain embodiments, assays of cell populations can be performed in a single reaction/tube or individually. By way of example but not limitation, measurement of co-expression of certain cell types can be performed independently and separately. In such embodiments, the inflammatory state of a subject can be evaluated based on individual and/or separate assays or the combined results of the assays. For example, when evaluation co-expression of CD16 or CD14 and CD86 or CD80, all four combinations of co-expression can be assessed or only one, e.g. CD16⁺/CD86⁺ can be assessed.

In certain embodiments, T cells and/or monocytes can be isolated or purified prior to culturing.

In any of the foregoing embodiments, the method may further comprise, if said subject is in an inflammatory state and said at least one cytokine is TNF-α: administering an anti-TNF-α therapy, in particular, a therapy that preferentially neutralizes the cell-free, soluble form of TNF-αwhile sparing the membrane-bound form of TNF-α, including but not limited to the recombinant receptor for TNF-α (etancercept; Enbrel®) or the anti-TNF-α monoclonal antibody adalimumab. By way of example but not limitation, these therapies may encompass those as described in Scallon B, Cai A, Solowski N, et al. Binding and Functional Comparisons of Two Types of Tumor Necrosis Factor Antagonists. Journal of Pharmacology and Experimental Therapeutics. 2002;301(2):418; Nguyen DX, Ehrenstein MR. Anti-TNF drives regulatory T cell expansion by paradoxically promoting membrane TNFTNF-RII binding in rheumatoid arthritis. The Journal of Experimental Medicine. 2016;213(7):1241. Specifically, in addition to etanercept and adalimumab, candidate anti-TNF-α agents that can be considered include: the etanercept biosimilars GP2015, CHS-0214, HD203, and SB4; and the adalimumab biosimilars ABP501, ZRC3197, CHS-1420, GP-2017, M923, SB5, FKB327.

In any of the foregoing embodiments, the method may further comprise, if said subject is in an inflammatory state and said at least one cytokine is IL-6: administering an anti-IL-6 therapy, potentially in combination with an anti-TNF-α therapy such as, by way of example but not limitation, etanercept, which will indirectly reduce IL-6 levels. In any of the foregoing embodiments, prior to collection of T cells by apheresis for input into T_{REG} manufacturing, the subject may be treated with one of a select group of anti-TNF-α reagents that preferentially alters the TCR repertoire for TNFR2-expressing input T cells that are shifted towards a T_{REG} phenotype. Alternatively, the said selective anti-TNF-α therapy can be followed by any other intervention designed to further promote T_{REG} cells in vivo, including but not limited to low-dose IL-2 therapy or rapamycin therapy. By way of example but not limitation, such low dose IL-2 therapy as described in Pham MN, von Herrath MG, Vela JL. Antigen-Specific Regulatory T Cells and Low Dose of IL-2 in Treatment of Type 1 Diabetes. Frontiers in Immunology. 2015; 6:651. or such rapamycin therapy as described in Hao Z, Miao M, Guo Y, et al. AB0536 Rapamycin attenuates symptom and restores the balance of th17/treg in refractory primary sjogren's syndrome. Annals of the Rheumatic Diseases. 2018;77(Suppl 2):1425. Specifically, IL-2 can be administered by subcutaneous injection daily for five consecutive days at doses ranging from 0.33 to 3.0×10⁶ IU/day. Specifically, rapamycin can be administered alone or in combination with IL-2 therapy, with rapamycin dosing adjusted in a patient-specific manner to yield effective serum trough concentrations ranging from 5 to 30 ng/ml.

In any of the foregoing embodiments, the method may further comprise, if said subject is in an inflammatory state and said at least one cytokine is IL-1β: administering an anti-IL-1β therapy. By way of example but not limitation, such IL-1β therapies may include the recombinant form of the naturally occurring IL-1 receptor antagonist, Anakinra; the soluble IL-1 decoy receptor, Rilanocept; and the monoclonal antibody Canakinumab. Such anti-IL-1β therapy can be administered alone or in combination with an anti-TNF-α therapy, which indirectly inhibits IL-1β.

In any of the foregoing embodiments, if said subject is in an inflammatory state, the method can further include administering nTREGS, manufactured TREGs (iTREGS) or a combination of both.

In some embodiments, the subject can be considered in an inflammatory state and therefore in need of immune modulation therapy if the RNA-based T cell receptor repertoire analysis and associated laboratory investigations indicate that the subject expresses an increase in T cell clonality relative to a control, particularly when found in association with an increased biologic drive towards T cell clonality. By way of example but not limitation, T cell receptor repertoire analysis may be performed as described in Rosati E, Dowds CM, Liaskou E, Henriksen EKK, Karlsen TH, Franke A. Overview of methodologies for T-cell receptor repertoire analysis. BMC Biotechnol. 2017;17(1):61. Specifically, purified RNA is transformed into a complementary determining region-3 (CDR3) library using multiplex polymerase chain reaction, target enrichment, or switch oligo nested polymerase chain reaction; subsequently, next generation sequencing is performed on the Illumina platform. It is preferable to use the Illumina HiSeq for deep sequencing to attain at least 300,000 on-target reads. Additionally, if there is insufficient normalization of such tests during a specific immune modulation therapy, then such a result should prompt an evaluation of an alternative dose of immune modulation or a change in immune modulation therapies. In some embodiments, the method can include isolating RNA or DNA from at least a portion of a sample comprising T cells from a subject suffering from ALS or isolating CD4⁺ and CD8⁺ T cell subsets from at least a portion of the sample to yield a nucleic acid sample, quantifying the TCR repertoire diversity in the nucleic acid sample, comparing the TCR repertoire diversity in the nucleic acid sample to a control sample or a standard value, where an increase in TCR repertoire diversity as indicated by an increase in the clonality index relative to the control sample or standard value is indicative that the subject is in an inflammatory state. In some embodiments, the increase can be at least 10%, 20%, 30%, 40%, 50% or 100%. In some embodiments, the control sample is a nucleic acid sample from a patient without ALS. In some embodiments, the standard value is a value associated with nucleic acid samples from patients without ALS. In some embodiments, the standard value is about 300,000 based on the CHAOE diversity index.

In some embodiments, the inflammatory state in ALS patients can be diagnosed and monitored by evaluating cyclic AMP response to CGS21680. In normal controls, who have abundant immune cell signaling through the adenosine A2A pathway, cAMP levels increase within 10 minutes and reach peak values at approximately two hours. In ALS patients, because there is down-regulation of the adenosine pathway, the cAMP curve is projected to be suppressed after CGS21680 exposure, with reduction of cAMP by at least 20% at the various time points of measurement from 10 minutes to two hours after exposure.

In any of the foregoing embodiments, the subject can already be receiving one or more therapies for ALS including, by way of example but not limitation, an anti-TNF-α therapy, an anti-IL-1β therapy, or an anti-IL-6 therapy. In some embodiments, the one or more therapies can be adjusted if the subject is in an inflammatory state. The adjustment can include treatment for the specifically elevated cytokine as described in the present disclosure. In some embodiments, the adjustment includes administering an additional therapy which can include, by way of example but not limitation, an anti-TNF-α therapy, an anti-IL-6 therapy, an anti-IL-1β therapy, or administration of nTREGS, manufactured T_{REG} cells (iTREGS) or a combination of both. In some embodiments, the additional therapy can include increase a dosage of the therapy the subject was already receiving.

In some embodiments, an anti-TNF-α therapy may be administered to the subject. By way of example but not limitation, such anti-TNF-α therapies may include the recombinant soluble receptor etanercept (Enbrel) or monoclonal antibody anti-TNF-α therapies that preferentially inhibit the soluble form of TNF-α for resultant selective inhibition of TNFR1-expressing Th1-type cells and preferential preservation of TNFR2-expressing T_{REG}-type cells.

In some embodiments, an IL-1β therapy may be administered to the subject. By way of example but not limitation, such IL-1β therapies may include the recombinant form of the naturally occurring IL-1 receptor antagonist, Anakinra; the soluble IL-1 decoy receptor, Rilanocept; and the monoclonal antibody Canakinumab. Such anti-IL-1β therapy can be administered alone or in combination with an anti-TNF-α therapy, which indirectly inhibits IL-1β.

In some embodiments, an anti-IL-6 therapy may be administered to the subject. By way of example but not limitation, such anti-IL-6 therapies may include the anti-IL-6 receptor monoclonal antibody, tocilizumab; and the anti-IL-6 monoclonal antibody, siltuximab. Such anti-IL-6 therapy can be administered alone or in combination with an anti-TNF-α therapy, which indirectly inhibits IL-6.

In any of the foregoing embodiments, the administration of a therapy can be performed using a therapeutically effective amount. By way of example, but not limitation, the therapy can include a therapeutically effective amount of an anti-TNF-α therapy, an anti-IL-6 therapy, an anti-IL-β therapy, or of nTREGS, manufactured TREGS (iTREGS) or a combination of both.

In some embodiments, where a patient is in an inflammatory state based on TRAF6, a pharmacologic inhibitor of TRAF6 such as, by way of example but not limitation, those described in Aarts S, Seijkens TTP, Kusters PJH, et al. Inhibition of CD40-TRAF6 interactions by the small molecule inhibitor 6877002 reduces neuroinflammation. J Neuroinflammation. 2017;14(1):105 can be used. It has been demonstrated in an animal model that this molecule can be used by systemic injection at a therapeutic dose of 10 μmol/kg recipient body weight to reduce neuroinflammation.

It should be understood that any of the diagnostic methods disclosed herein, can be combined in any combination to provide a immune diagnostic panel. For example, an immune diagnostic panel can include determining inflammation by the (1) co-culture of T cells and monocytes; co-culture of T cells and monocytes in the presence of an anti-PDL1 or anti-PD1 antibody or fragment thereof of anti-CD200L or anti-CD200 antibody or fragment thereof; and RNA-based T cell receptor sequencing, as described in the present disclosure.

### Examples

The following examples are provided to better illustrate the methods of the present disclosure. These examples are not intended to be limiting or to otherwise alter the scope of the methods disclosed in the present disclosure.

### Example 1: Inflammatory Monitoring in ALS Patients: Flow Cytometry

Previous studies using flow cytometry to monitor ALS patients indicate that: (1) quantification of nT_{REG} cell number is not an extremely robust indicator of ALS severity, including when assessing methods that incorporate CD4 cell expression of the T_{REG} transcription factor FoxP3; rather, investigations have relied upon other methods such as epigenetic analysis or functional assays; (2) there is a paucity of existing data regarding the use of flow cytometry to assess the Th1 transcription factor, T-bet; and (3) there is a paucity of information pertaining to the induced, post-thymic component of TREGS (T_{REGS}), including both CD4⁺ and the less well characterized CD8⁺ subsets of iT_{REGS}.

In response, we have developed a multi-color flow cytometry assay that combines surface markers with intra-cellular analysis of both T_{REG} and Th1 transcription factors (FoxP3 and T-bet, respectively). Co-expression of transcription factors has been described in other settings and is consistent with T cell plasticity towards various differentiation states; as such, T cells that co-express the markers FoxP3 and T-bet are operationally-defined as transitional cells with differentiation plasticity rather than bona-fide, committed T_{REG} cells. In a similar manner, we have applied a method of co-expression of FoxP3 with intra-cellular T cell inflammatory cytokines IL-2 and IFN-γ to also differentiate bona-fide T_{REG} populations from transitional cells; that is, in a manner similar to that described by others, we define ALS patient T cells that co-express FoxP3 and either IL-2 or IFN-γ to be non-T_{REG} cells. Of note, it has long been observed that inflammatory, effector T cells in humans can transiently express FoxP3. On the other hand, we define singular expression of FoxP3 in the absence of T-bet, IL-2, or IFN-γ as a regulatory population, including whether this pattern is observed on the CD4, CD8, natural, or inducible subsets based on cell surface staining.

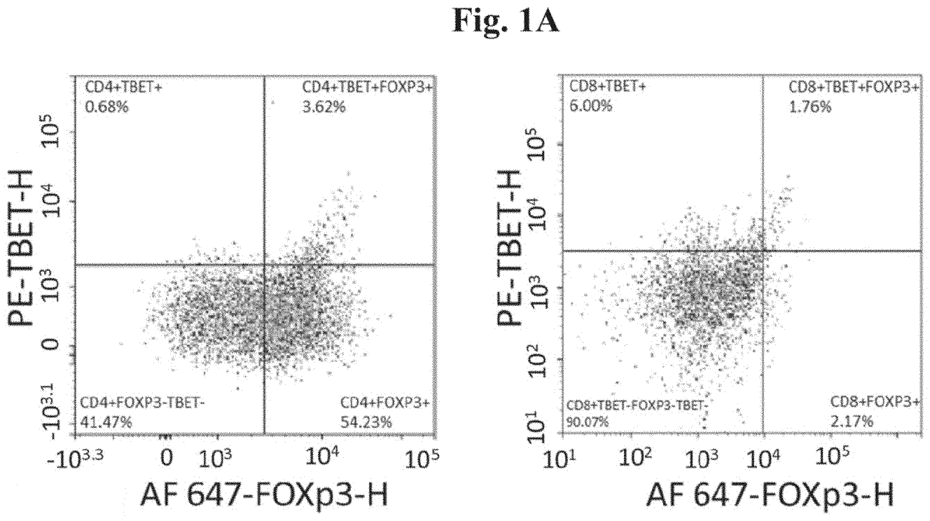

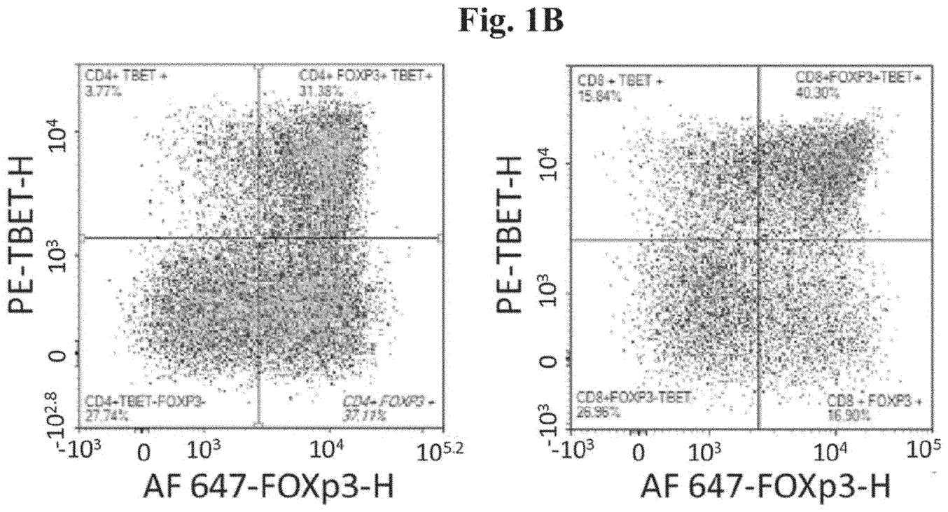

FIGS. 1A-1B show a representative approach of this method of detecting co-expression of FoxP3 with non-T_{REG} molecules such as T-bet, IL-2, or IFN-γ. In FIGS. 1A-1B, peripheral blood mononuclear cells from a normal control (data shown in FIG. 1A) and an ALS patient (data shown in FIG. 1B) were subjected to surface flow cytometry and intra-cellular flow cytometry for both the T_{REG} and Th1 transcription factors, FoxP3 and T-bet, respectively. In FIG. 1A, flow cytometry of the peripheral blood mononuclear cells from a normal control, both CD4⁺ T cells (gated cells shown in left panel) and CD8⁺ T cells (gated cells shown in right panel) had primarily T cells expressing only one transcription factor. Specifically, 54.2% of CD4 cells expressed FoxP3 whereas only 3.6% expressed both FoxP3 and TBET; furthermore, only 6.0% of CD8 cells expressed TBET and only 1.8% of CD8 cells expressed both FoxP3 and TBET. In marked contrast, in FIG. 1B, flow cytometry of the peripheral blood mononuclear cells from an ALS patient, within the CD4 population (left panel), there were significant subsets of both single positive FoxP3 cells (37.1%) and cells double-positive for both FoxP3 and TBET (31.4%). Similarly, within gated CD8 cells from the ALS patient, there were significant subsets of both single positive FoxP3 cells (16.9%) and double-positive FoxP3⁺ and TBET⁺ cells (40.3%). T cells that co-express transcription factors have enhanced differentiation plasticity, and therefore are consistent with an inflammatory profile.

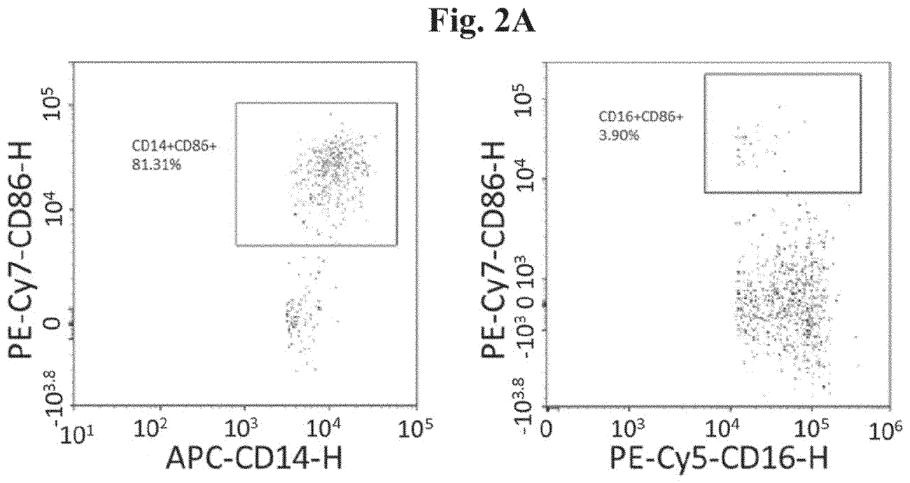

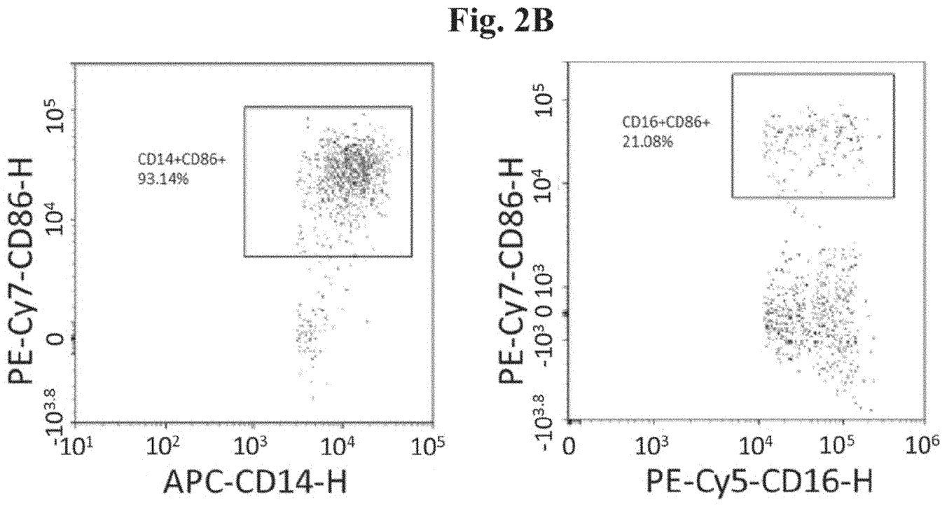

Monocyte flow cytometry can also be used to monitor the inflammatory state of ALS. In previous studies, the monocyte inflammatory status in ALS was measured by RNA analysis; in a separate study, ALS patients had an increase in CD14⁺ monocyte expression of the co-stimulatory molecules CD80 and CD86, which contribute to the pathogenesis of auto-immune disease. As such, monocyte expression of CD80 and CD86 represents a functional marker of inflammatory potential that will supplement the other tests that we have developed. FIGS. 2A-2B show increased monocyte expression of CD86 in an ALS patient (FIG. 2B) relative to a normal control (FIG. 2A). Within the CD14⁺ monocyte subsets (left panels of FIGS. 2A-2B), the ALS patient had 93.1% of cells expressing CD86 whereas the control had only 81.3% of cells expressing CD86. In marked contrast, within the CD16⁺ population of monocytes, the ALS patient had 21.1% of cells expressing CD86, which was greatly increased relative to the 3.9% expression of CD86 on the normal control monocytes. As such, it is critical to monitor ALS patient monocyte inflammatory status by CD86 measurement on both CD14⁺ and CD16⁺ monocyte subsets. The increase in CD86 was primarily observed within the CD16⁺ subset of monocytes, which was not previously emphasized in ALS patients. As such, monitoring of CD86 expression in the CD16⁺ monocyte subset represents a new tool for monitoring inflammation in ALS patients.

Other techniques that can be used in corresponding methods include single cell Western blot and ELISPOT.

### Example 2: Inflammatory Monitoring in ALS Patients: Autonomous Cytokine Secretion

Ex vivo cytokine secretion in the resting state (without antigenic or pharmacologic provocation) is a sign of in vivo immune activation. For example, normal controls secrete very low levels of the inflammatory cytokines IL-6 and TNF-α under resting conditions, consistent with negligible autonomous cytokine secretion. There is a paucity of data in the literature relating to the spontaneous production of cytokines in cells from ALS patients that are comprised of both T cells and monocytes; rather, the field in general has emphasized measurement of serum or CSF cytokines, which may not reflect cellular events in the micro-environment of interest.

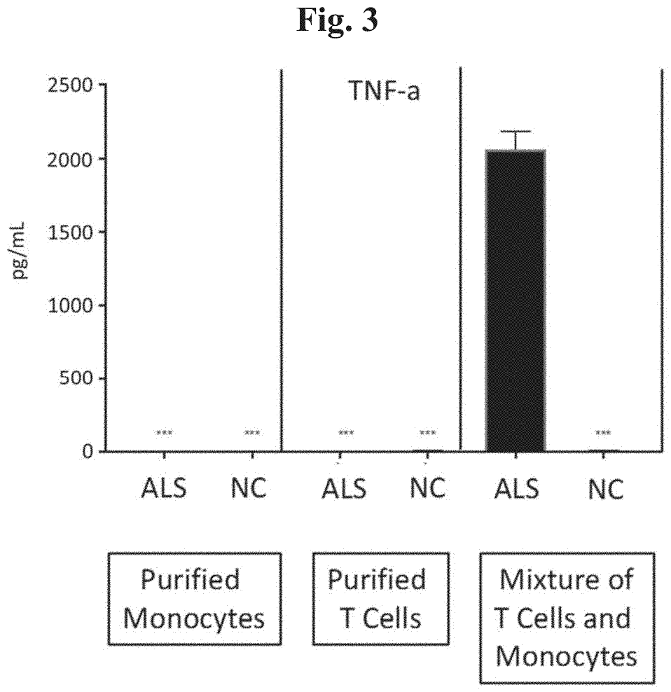

We have observed that ALS patients can manifest autonomous inflammatory cytokine secretion when incubation of T cell- and monocyte-containing inoculum occurs for 24 hours in X-Vivo 20 media supplemented only with 5% human serum (FIG. 3). FIG. 3 depicts ALS patient and normal control autonomous cytokine secretion. ALS patient (“ALS”) and normal control (“NC”) peripheral blood mononuclear cells were either not separated (FIG. 3, right panel; mixture of T cells and monocytes), or separated into CD14⁺ monocyte cells (FIG. 3, left panel) or CD3⁺ T cells (FIG. 3, middle panel). The cells were incubated for 24 hours in media without any stimulation additives. After 24 hours, the supernatant was harvested and tested for cytokine content; TNF-α content is shown, with result described as pg per ml per 1×10⁶ cells per 24 hours. A similar result was observed for content of IL-1β and for IL-6. Of note, the autonomous inflammatory cytokine secretion was only observed when the two populations were co-cultured; this result is consistent with a mechanism of immune activation in ALS patients whereby T cell help is provided to monocytes for inflammatory cytokine secretion. As such, optimal monitoring of inflammatory status in ALS patients should necessarily include a mixture of T cells and monocytes rather than purified singular populations; this cell mixture may occur in the distribution that occurs naturally in vivo or may be controlled at different ratios to better characterize T cell and monocyte interactions. We reason that such help might be provided by CD40 ligand (CD154), which is a prominent mediator of T cell help. CD40L is also implicated in the pathogenesis of experimental ALS; furthermore, more than 50% of ALS patients had a general up-regulation of genes in the CD40 pathway when PBMC were evaluated by RNA analysis of an array of genes.

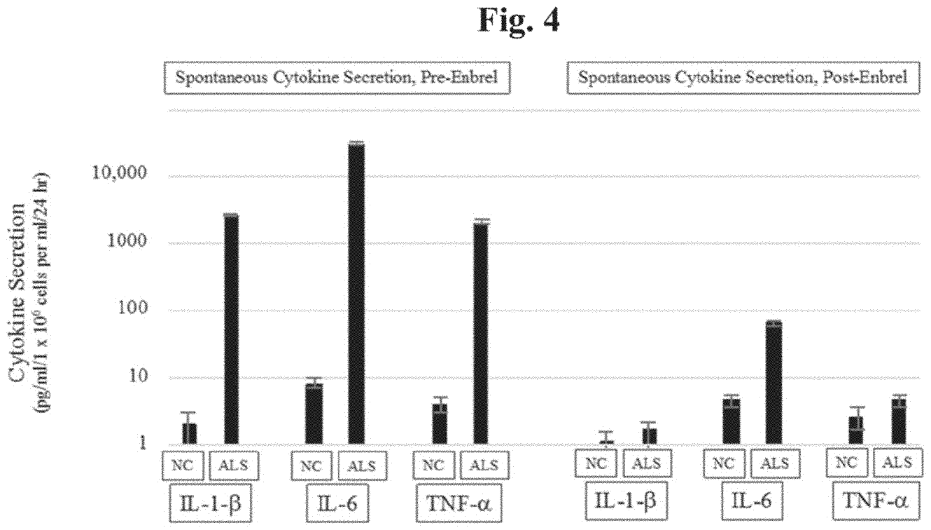

We reasoned that detection of autonomous secretion of inflammatory cytokines will have potential clinical implications, for example, in the monitoring of ALS disease response to investigational therapy. Towards this aim, we evaluated cytokine secretion in an ALS patient prior to and then after experimental therapy with the anti-TNF-α soluble recombinant receptor, etancercept (Enbrel®); there have been no reports of clinical trials evaluating etanercept therapy of ALS. As shown in FIG. 4, anti-TNF therapy reduced autonomous secretion of IL-1-13, IL-6, and TNF-α. In FIG. 4, peripheral blood mononuclear cells were collected from a 58-y.o. male with ALS and a normal control (NC). As depicted in FIG. 4, left panel, cells were incubated for 24 hr. without any stimulation and the supernatant was tested for cytokine content by multiplex assay (values shown are in pg/ml per 1×10⁶ cells per 24 hr). As depicted in FIG. 4, right panel, The ALS patient was started on etanercept therapy, 50 mg per week by subcutaneous injection. A similar result was obtained on three consecutive values spanning three months during etanercept therapy. In addition to demonstrating the value of our cytokine measurement assay, the results demonstrate that TNF-α can act as not only a distal effector cytokine in ALS pathogenesis, but also as a driver-cytokine that can work more proximally in the cytokine cascade; in this manner, etanercept can control a diversity of inflammatory pathways implicated in ALS pathogenesis, namely IL-1-β and IL-6.

### Example 3: Inflammatory Monitoring in ALS Patients: CD40 Ligand-driven Cytokine Secretion

We reasoned that some ALS patients may not secrete inflammatory cytokines autonomously due to a relative deficiency in CD40 ligand (CD40L), which may occur if activated T cells are trafficking into the tissue site (CNS), if activated T cells are deleted in the CNS due to antigen-induced cell death (AICD), or if activated T cells receive tolerance signals through the PD1 pathway or other checkpoint inhibitory pathways.

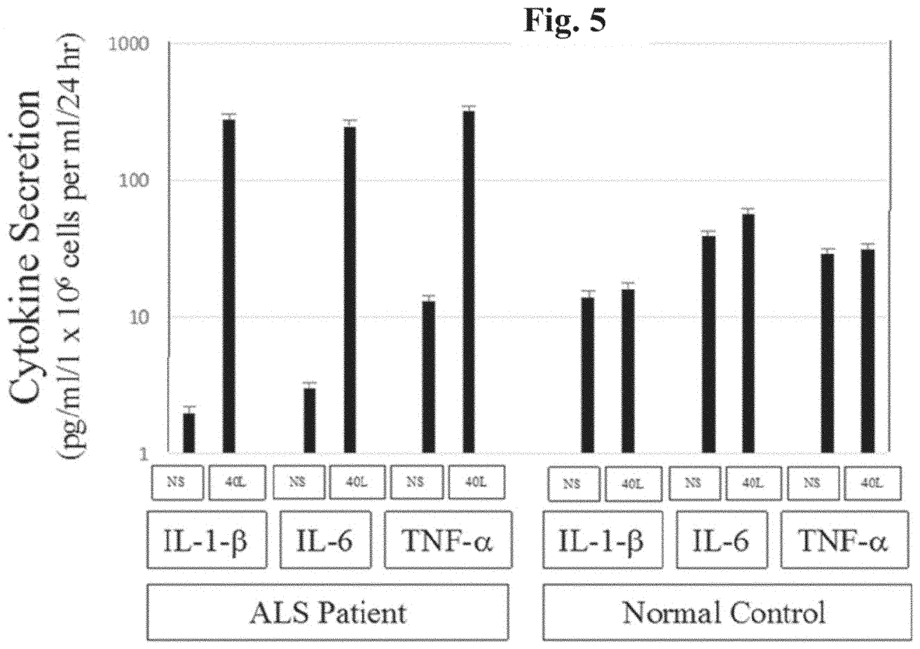

Given this possibility, we evaluated whether simple addition of the recombinant human (rhu) CD40L molecule might uncover autonomous inflammatory cytokine secretion in ALS patients. There are no reports in the literature to date pertaining to CD40L-stimulation of ALS patient PBMC. As FIG. 5 demonstrates, addition of rhu CD40L can induce inflammatory cytokine production in ALS patients. In FIG. 5, PBMC from an ALS patient (data shown in left panels) and a normal control (data shown in right panels) were placed into culture for 24 hours in X-Vivo 20 media supplemented with 5% human serum (no stimulation; “NS”); in parallel, the same cells were incubated but with the addition of recombinant human CD40 ligand (“40L”). After 24 hours, the supernatants were harvested and tested for cytokine content by multiplex assay (results expressed as pg/ml per 1×10⁶ cells per ml per 24 hours). Therefore, the detection of the inflammatory subset of ALS can be expanded by inclusion of patients that secrete cytokines autonomously and those patients that secrete inflammatory cytokines after CD40L stimulation. Patient hypersensitivity to CD40 ligand is driven by an imbalance in the post-CD40 receptor signaling pathway, with increased TRAF6 contribution to the signaling cascade during neuro-inflammation; importantly, the TRAF2 and TRAF3 family members inhibit post-CD40 signaling. As such, given this information, our monitoring method will also include Western Blot analyses for the detection of ALS patient balance of the TRAF family of proteins.

ALS is a disease that is familial in about 10% of patients, with many different inherited mutations responsible for this disease predisposition. However, the penetrance of this disease inheritance, such as that which occurs with the C9orf72 mutation, is widely distributed across the age range spectrum. As such, interventional trials will be difficult to implement in the carrier population due to the widely varying timing of disease onset. Given this obstacle, there exists a great need to define other factors in addition to mutational status that will help determine disease risk. We propose that an inflammatory phenotype will represent one such additional risk factor. Of interest, individuals with inherited aspects of the CD40 pathway are at-risk for development of a variety of auto-immune diseases; as such, we project that mutation-carrier patients with evidence of hyper-responsiveness of the CD40 pathway will be at heightened risk for ALS disease development, and therefore will be appropriate candidates for early intervention trials.

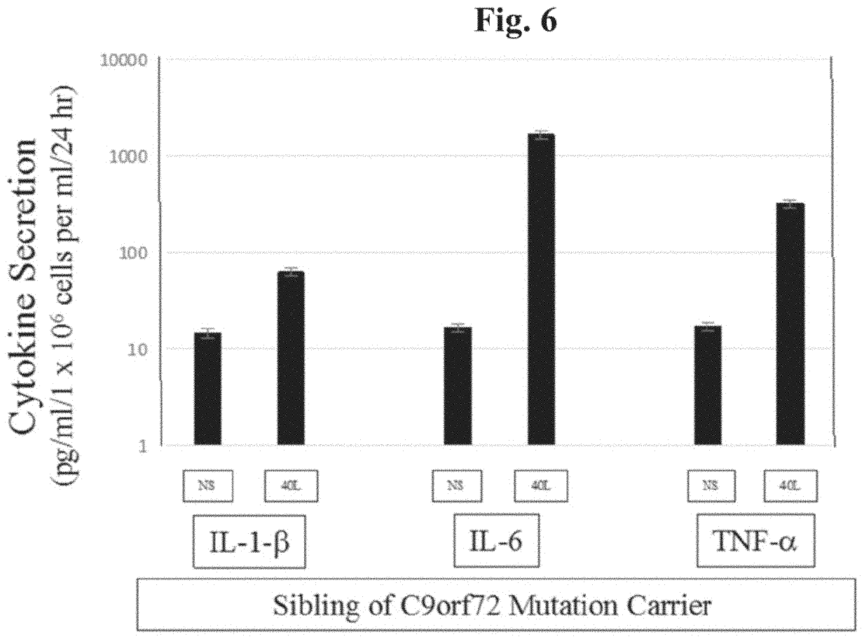

Consistent with this idea, we found that siblings of a C9orf72 carrier can manifest the inflammatory cytokine phenotype, as evidenced by high levels of cytokine secretion after stimulation with CD40L (FIG. 6). In FIG. 6, a sibling of a subject who was a carrier for the C9orf72 mutation was evaluated for autonomous cytokine secretion. PBMC were cultured either in X-Vivo 20 media supplemented with 5% human serum (no stimulation; “NS”) or the same media with the further addition of recombinant human CD40 ligand (“40L”). Supernatants were collected after 24 hours and tested for cytokine content by multiplex assay (results expressed as pg/ml per 1×10⁶ cells per ml per 24 hours). This illustrates the importance of both neurologic and immunologic risk factors. As such, screening of individual family members for CD40 pathway hyper-responsiveness represents a method for risk stratification in subjects carrying ALS-associated genes: that is, individuals with both the genetic risk factor and the immunologic signal of increased CD40L responsiveness will be at imminent risk of disease development.

### Example 4: Inflammatory Monitoring in ALS Patients: Adenosine Receptor-modulated Cytokine Secretion

Adenosine is a natural product that can play divergent roles in the immune system, including as it relates to neuro-inflammation. In general, adenosine signaling through the adenosine A2_{A} receptor provides an anti-inflammatory signal; in contrast, adenosine signaling through the adenosine A3 receptor provides a pro-inflammatory signal. As such, adenosine receptor distribution or variation might represent one factor that controls the inflammatory state. Given this information, we reasoned that the use of pharmacologic agonists and antagonists to the adenosine A2_{A} and A3 receptor during the autonomous cytokine secretion assay might represent an additional tool to unmask inflammation in ALS patients.

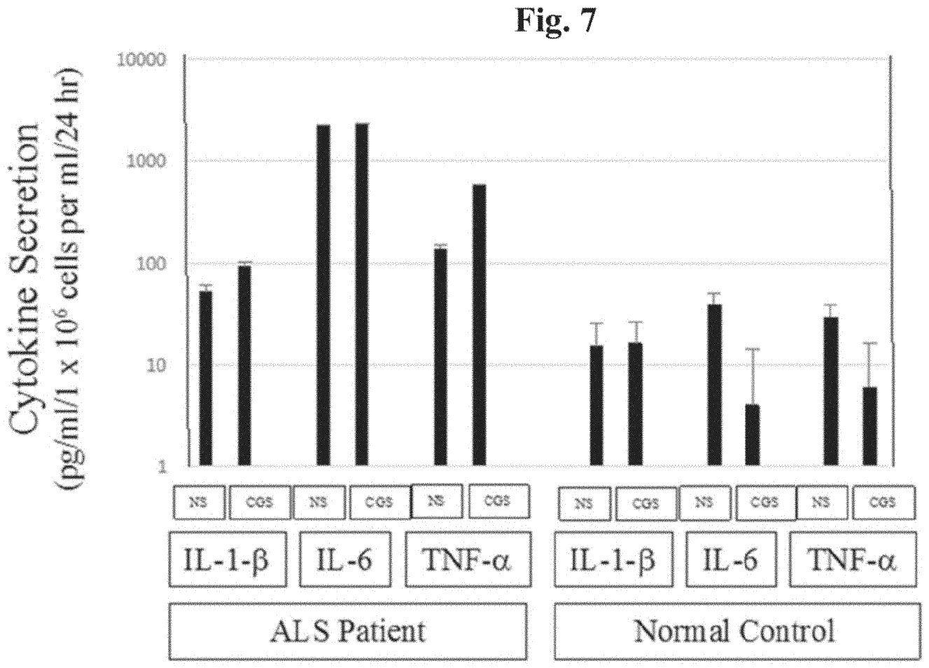

Indeed, as FIG. 7 demonstrates, addition of the adenosine A2_{A} receptor agonist CGS21680 can increase ALS patient inflammatory cytokine secretion (in this experiment, increase in IL-1-β and TNF-α). In FIG. 7, PBMC from an ALS patient (FIG. 7, left panels) and a normal control (FIG. 7, right panels) were placed into culture for 24 hours in X-Vivo 20 media supplemented with 5% human serum (no stimulation; “NS”); in parallel, the same cells were incubated in media plus the addition of the adenosine A2A receptor agonist CGS21680 (“CGS”). After 24 hours, the supernatants were harvested and tested for cytokine content by multiplex assay (results expressed as pg/ml per 1×10⁶ cells per ml per 24 hours). There have been no reports in the literature pertaining to the use of adenosine receptor modulation for characterization of the inflammatory state in ALS patients.

### Example 5: Inflammatory Monitoring in ALS Patients: Programmed Death 1 and Other T Cell Checkpoint Pathway Modulated Cytokine Secretion

T cells can be further suppressed by members of the checkpoint inhibitor family; most notably, T cell expression of programmed death-1 (PD1) receptor can result in T cell suppression if the PD1 receptor is ligated by programmed cell death ligand-1 or -2 (PDL1, PDL2). In the periphery, otherwise reactive T cells might have plentiful interaction with PDL1/PDL1 and thereby may exist in the suppressed state that is undetectable using conventional methods. However, such T cells, when infiltrating into the CNS, may find very limited amounts of PDL1/PDL2; in such a case, the T cells are liberated from their suppression and become activated T cells that can contribute to neuro-inflammation.

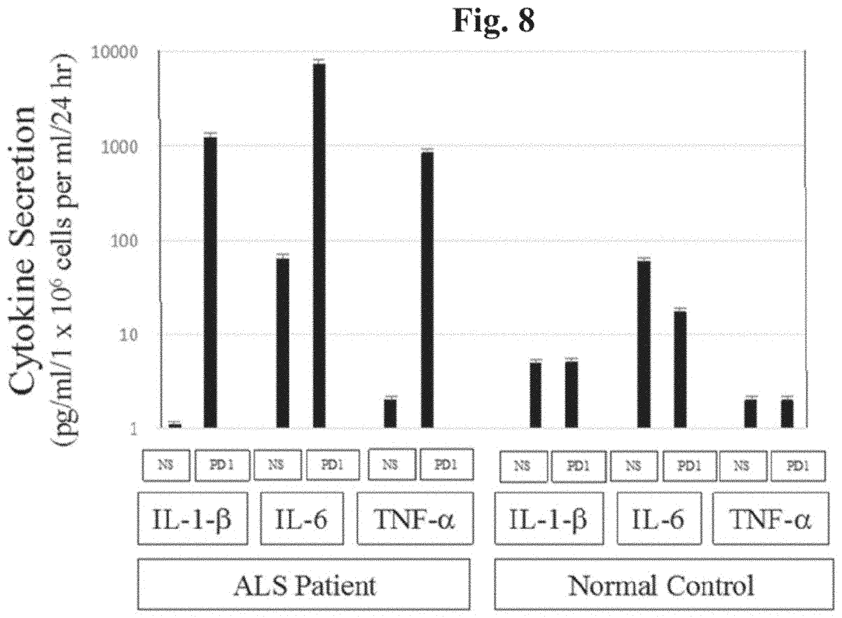

To mimic this potential biology, we performed the autonomous cytokine secretion assay in the presence of a blocking antibody to PDL1, thereby removing any potential inhibitory PD1 signaling. In FIG. 8, PBMC from an ALS patient (FIG. 8, left panels) and a normal control (FIG. 8, right panels) were placed into culture for 24 hours in X-Vivo 20 media supplemented with 5% human serum (no stimulation; “NS”); in parallel, the same cells were incubated in media plus the addition of a neutralizing antibody to PDL1 (“PD1”). After 24 hours, the supernatants were harvested and tested for cytokine content by multiplex assay (results expressed as pg/ml per 1×10⁶ cells per ml per 24 hours). As FIG. 8 shows, removal of the PD1 signaling pathway in ALS patient T cells indeed can result in the unmasking of inflammation, as evidenced by the spontaneous secretion of inflammatory cytokines. In addition to removal of the PD1 checkpoint inhibitor, the unmasking of inflammation in ALS patients may be further accomplished through the incorporation of other T cell checkpoint inhibitors into the assay, including but not limited to anti-CTLA4 and anti-TIM3.

### Example 6: Inflammatory Monitoring in ALS Patients: CD200 and Other Monocyte Checkpoint Pathway Modulated Cytokine Secretion

Monocytes are also subjected to checkpoint inhibition, particularly through the CD200 and CD47 pathways. Of note, insufficient monocyte checkpoint inhibition is associated with predisposition to autoimmune disease. Monocytes that are ligated through the CD200 receptor are suppressed; potentially, monocytes may have ready access to CD200 receptor ligands in the periphery but restricted access to such ligands in the CNS, thereby resulting in loss of monocyte checkpoint inhibition and subsequent exacerbation of neurodegeneration. As such, we propose that use of blocking agents to the CD200 pathway may represent an additional in vitro diagnostic tool to unmask the inflammatory state in ALS patients.

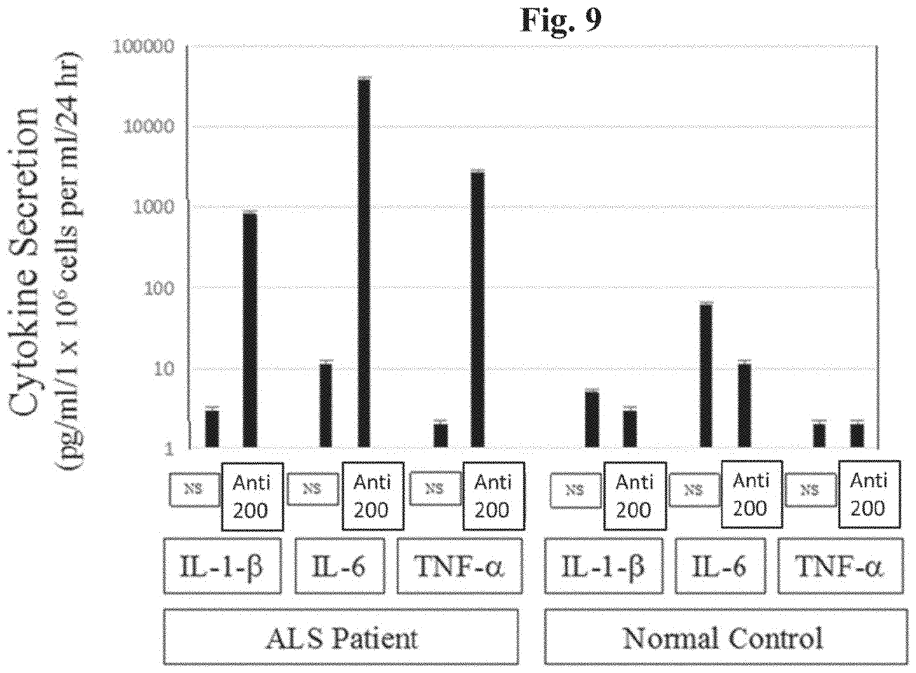

In FIG. 9, PBMC from an ALS patient (FIG. 9, left panels) and a normal control (FIG. 9, right panels) were placed into culture for 24 hours in X-Vivo 20 media supplemented with 5% human serum (no stimulation; “NS”); in parallel, the same cells were incubated in media plus the addition of a neutralizing antibody to CD200 ligand (“anti-200”). After 24 hours, the supernatants were harvested and tested for cytokine content by multiplex assay (results expressed as pg/ml per 1×10⁶ cells per ml per 24 hours). As demonstrated in FIG. 9, blockade of the CD200 pathway in ALS patient PBMC samples can result in the unmasking of inflammatory cytokine secretion. Additional monocyte checkpoints may also be unmasked through this approach, including but not limited to blockade of the CD47 pathway.

### Example 7: Inflammatory Monitoring in ALS Patients: T Cell Receptor (TCR) Repertoire

Although there is consensus that peripheral T cells can infiltrate the CNS and contribute to ALS pathogenesis, there exists very limited understanding in terms of the specific antigens that are recognized by the antigen-specific T cells that are thought to propagate the disease. Improving an ability to detect the specific T cell response in ALS patients will be beneficial from the perspectives of diagnosis, risk stratification, and treatment monitoring. That is, it is possible that patients with higher numbers of immune T cell clones that are present at increased frequencies and potentially reactive to CNS tissue will have an adverse prognosis; in a similar vein, therapeutic interventions that help normalize this skewed T cell repertoire will be advantageous both in terms of limiting neuro-inflammation and providing protection against infection, which represents a considerable cause of mortality in ALS patients.

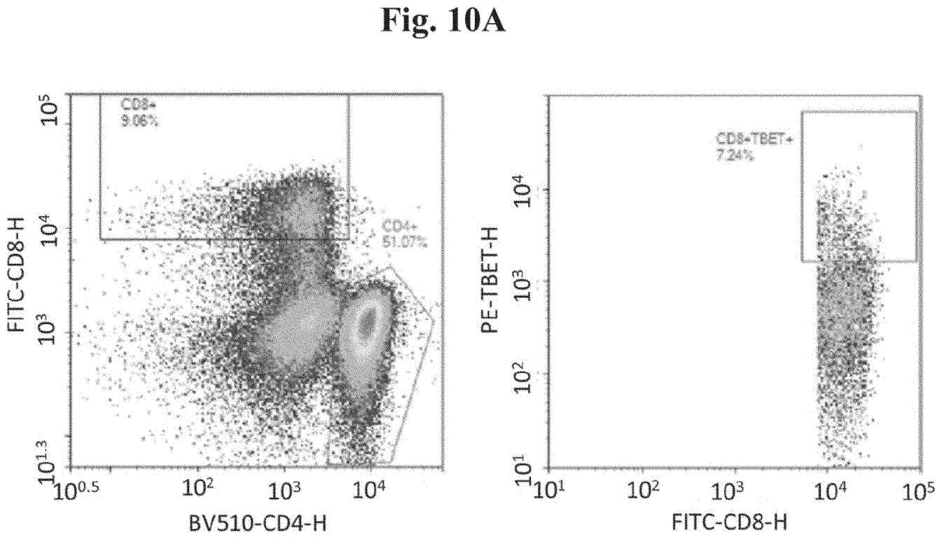

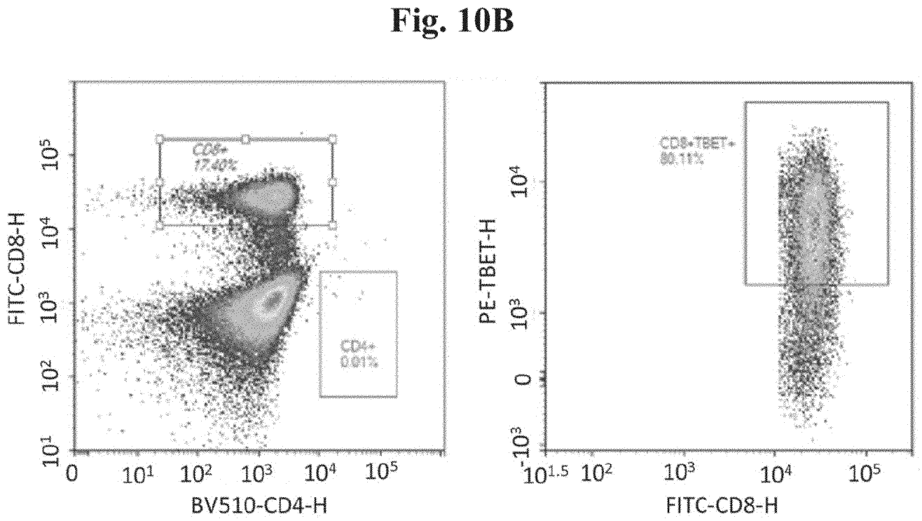

In addition to the identification of clonally expanded T cells that may be reactive to CNS antigens, it is also possible that initially activated clones may be deleted in the inflammatory milieu through a process known as antigen-induced-cell-death (AICD). In some clinical settings, including HIV infection, AICD is thought to contribute to a reduction in T cell counts (in particular, CD4⁺ T cells) and to predispose to opportunistic infection. Consistent with this biology, we have observed that an extreme reduction in CD4⁺ T cells in a patient with ALS who expressed an inflammatory cytokine phenotype and a rapidly progressive clinical course (FIGS. 10A-10B). Peripheral blood mononuclear cells from a normal control (FIG. 10A) and an ALS patient (FIG. 10B) were evaluated by flow cytometry for distribution of CD4⁺ and CD8⁺ T cell subsets (left panels) and for expression of the Th1-type transcription factor TBET within the CD8⁺ T cell subset (right panels). FIG. 10A shows that the normal control had a fairly typical CD4-to-CD8 proportion (51.1% and 9.1% of T cells) whereas FIG. 10B shows that the ALS patient had greatly diminished contribution from the CD4⁺ T cell subset (CD4⁺ and CD8⁺ contributions of 0.01% and 17.4%, respectively). In addition, in the normal control, the CD8⁺ T cells had a fairly typical level of TBET transcription factor expression, namely, 7.2% of the CD8 cells were TBET⁺; in marked contrast, in the ALS patient, 80.1% of the CD8 cells were TBET⁺. These data indicate that the inflammation associated with ALS can induce an AICD-like pattern of CD4 cell depletion.

In ALS patients, we therefore reason that an analogous AICD phenomenon may occur due to exposure of T cells to a broad array of CNS antigens in a highly inflammatory environment; in such cases, the T cell receptor (TCR) repertoire will be further skewed because both clonally expanded and clonally deleted events will be captured. Towards this aim, we have incorporated state-of-the-art TCR sequencing into our array of tests to assess the inflammatory status of ALS patients. Although DNA sequencing of the TCR repertoire is often utilized, our method specifies the preferential utilization of RNA sequencing of the TCR repertoire, which has been shown to be potentially advantageous in terms of increased sensitivity, reduced errors, and ability to accurately identify alternative splicing events.

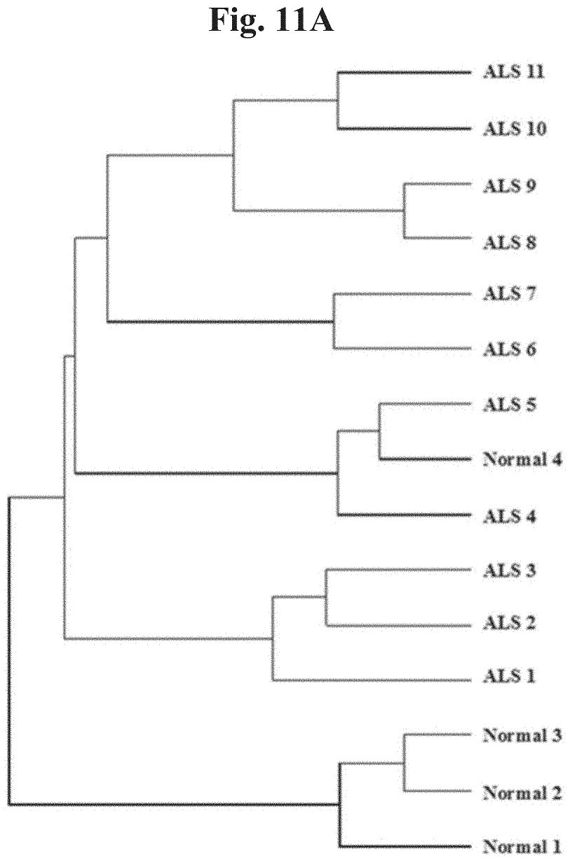

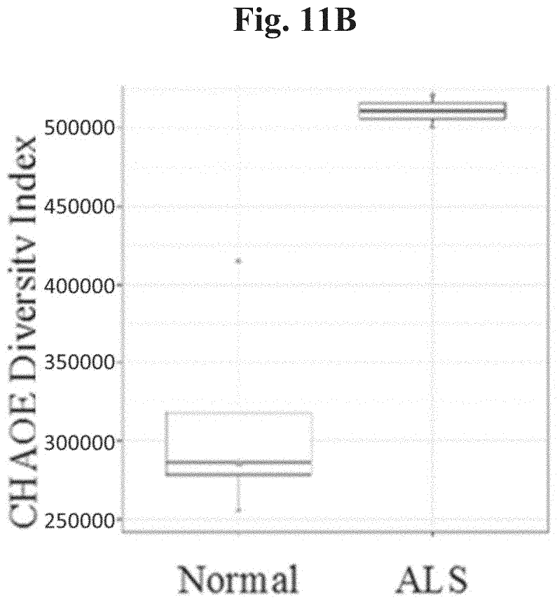

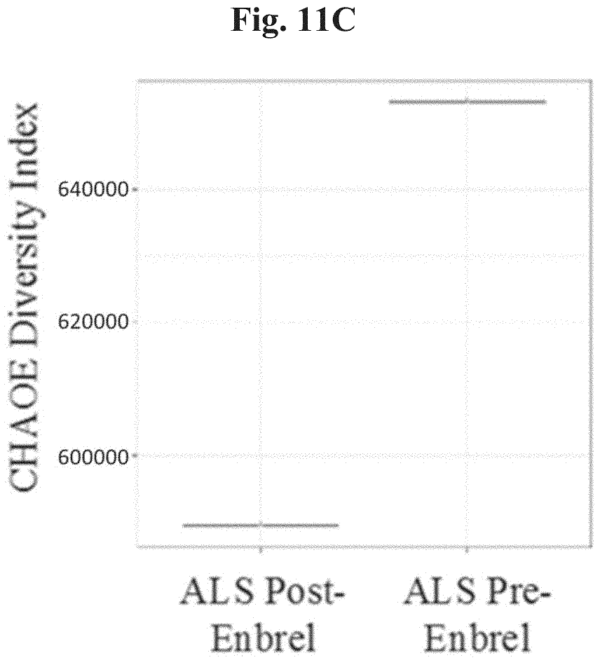

FIGS. 11A-11C depict the use of RNA-based T cell receptor sequencing to differentiate normal controls from ALS subjects and to monitor ALS patient response to immune modulation therapy. RNA was isolated from peripheral blood mononuclear cells from normal controls (n=4) and from ALS subjects (n=11). The RNA was subjected to TCR repertoire profiling, as previously described. FIG. 11A depicts a family tree global TCR repertoire analysis is detailed. These data indicate that this tool can segregate ALS samples from normal control samples. As indicated in FIG. 11B, the TCR clonality was quantified in n=4 normal control samples and n=2 ALS samples using the CHAOE diversity index. These data indicate an approximate 66% increase in TCR clonality in the ALS samples (an increase from ˜300,000 to −500,000). The analysis also indicated that the majority of the increased clonality in the ALS samples was attributable to alternative splicing. As indicated in FIG. 11C, the TCR clonality in an ALS patient after immune therapy with Enbrel was partially normalized relative to the pre-therapy sample, as indicated by reduction in the Diversity Index from approximately 650,000 to approximately 599,000.

Using a global family tree gene expression analysis, TCR profiling by RNA sequencing analysis was able to discriminate normal control samples from samples obtained from ALS patients (FIG. 11A).

TCR profiling by RNA sequencing analysis identified that ALS patient T cells have a large increase in T cell clonality (FIG. 11B), which in other analyses that we performed was attributable in large part to an increase in alternative splicing. The magnitude of T cell clonality increase in ALS subjects (˜66%) was higher than that described in other diseases such as auto-immune celiac disease, in which an ˜6% increase in T cell clonality has been estimated.

Because of this newly discovered high frequency of TCR clones generated through alternative splicing, we now propose that ALS and other neurodegenerative diseases can be diagnosed and monitored by assessment of alternative splicing events, including but not limited to T cell expression of global regulators of splicing in activated T cells such as hnRNPLL and measurement of up to 132 alternatively spliced genes as described in Oberdoerffer S, Moita LF, Neems D, Freitas RP, Hacohen N, Rao A. Regulation of CD45 alternative splicing by heterogeneous ribonucleoprotein, hnRNPLL. Science. 2008; 321(5889):686-691. Such alternatively spliced genes include but are not limited to: ADAM metallopeptidase domain 15 (metargidin); interleukin 4 induced 1; signal transducer and activator of transcription 5A; TNF receptor-associated factor 1; and sirtuin 5.

TCR profiling also identified that the increased clonality in ALS patient T cells was partially normalized after immune modulation therapy with etanercept, as indicated by an approximate 10% reduction in clonality in the post-therapy sample (FIG. 11C).

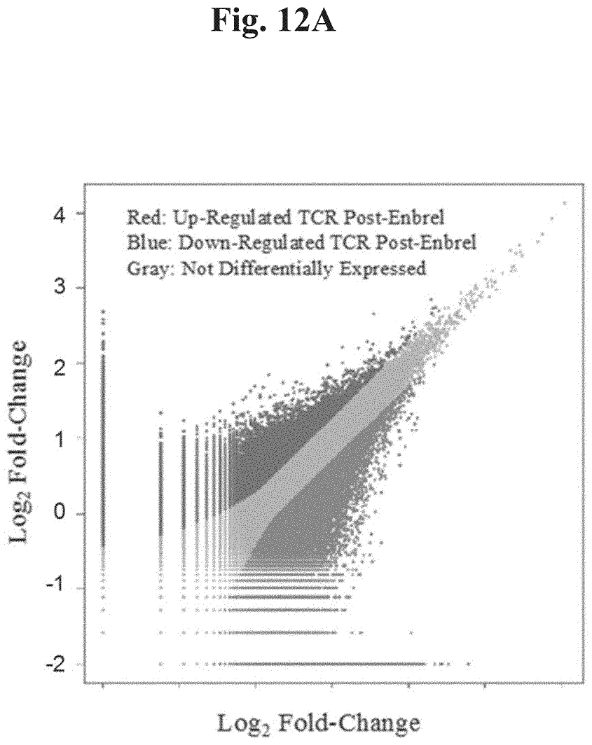

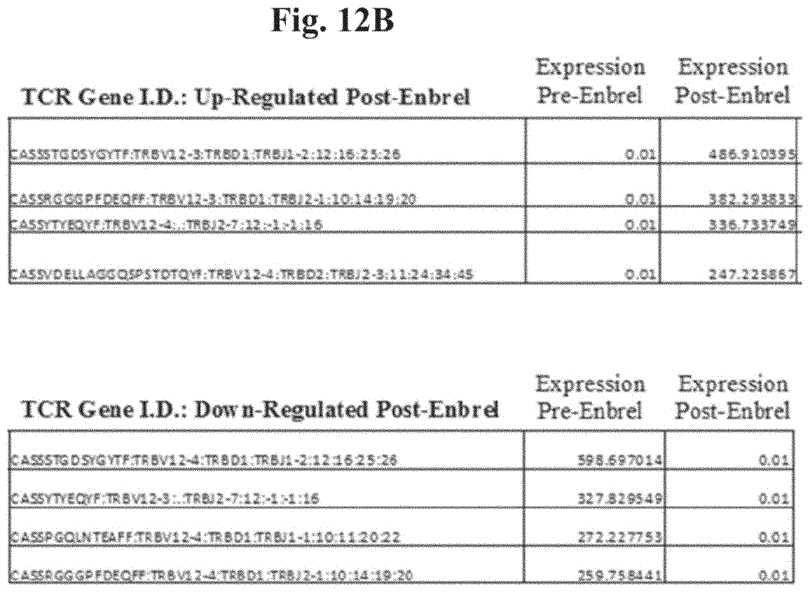

FIGS. 12A-12B depict use of RNA-based T cell receptor sequencing to monitor ALS patient response to immune modulation therapy. RNA was isolated from peripheral blood mononuclear cells from an ALS patient pre- and post-therapy with etanercept therapy. The RNA was subjected to TCR repertoire profiling, as previously described. In FIG. 12A, it is demonstrated that approximately 25% of TCR specificities were up-regulated in the post-therapy sample (as indicated in red); in marked contrast, approximately 25% of TCR specificities were down-regulated in the post-therapy sample (as indicated in blue). As indicated in the upper right of FIG. 12B, etanercept therapy resulted in marked T cell clonal expansion, as several T cell clones increased from frequencies of 0.01 pre-etanercept (near the detection limit of the assay) to post-treatment values ranging from 247 to 486, thereby consistent with a more than 4-log T cell expansion. As indicated in the lower right of FIG. 12B, etanercept therapy resulted in marked T cell clonal contraction, as several T cell clones decreased from frequencies of 259 to 598 pre-etanercept to post-treatment values of 0.01, thereby consistent with a more than 4-log T cell clonal contraction.

TCR repertoire analysis by RNA sequencing also identified that immune modulation therapy with etanercept resulted in a large-scale change in the TCR repertoire, with the post-therapy sample having reduced expression of −25% of the TCR sequences (FIG. 12A; reduced clones indicated in blue) and increased expression of −25% of the TCR sequences (FIG. 12A; increased clones indicated in red).

As FIG. 12B (lower panel) indicates, the down-regulation of specific TCR clones due to etanercept therapy was not only global (25% of repertoire affected) but also marked within individual clones. That is, individual clonal frequencies were reduced from values well above 100 to values close to the detection limit (0.01), thereby indicated an approximate 4-log reduction in clonal frequency consistent with clonal deletion.

As FIG. 12B (upper panel) indicates, the up-regulation of specific TCR clones due to etanercept therapy was not only global (25% of repertoire affected) but also marked within individual clones. That is, individual clonal frequencies were increased from values close to the detection limit (0.01) to values greater than 100, thereby indicated an approximate 4-log increase in clonal frequency consistent with marked clonal expansion.

These data indicate that etanercept markedly alters the T cell receptor repertoire during neurodegenerative disease (ALS), an observation that has not been previously documented.

This marked alteration in the TCR repertoire due to etanercept is predictably due to the following series of observations: etanercept preferentially binds to the soluble form of TNF-α rather than the membrane-bound form of TNF-α; the soluble form of TNF-α preferentially binds to and promotes T cells that express the type 1 TNF-α receptor (TNFR1), which are primarily Th1 cells; and the transmembrane form of TNF-α preferentially binds to and promotes T cells that express the type 2 TNF-α receptor (TNFR2), which are primarily T_{REG} cells. Taken together, we conclude that etancercept and other therapeutics such as select anti-TNF-α monoclonal antibodies that preferentially target the soluble form of TNF-α represent effective modalities to beneficially alter the TCR repertoire by reducing TNFR1-expressing Th1 cells and augmenting TNFR2-expressing T_{REG} cells in patients with neurodegenerative disease such as ALS.

According to these observations, the efficacy of any therapeutic to beneficially shift the immune TCR repertoire to a protective phenotype in subjects with neurodegenerative disease can be quantified by assessing the magnitude of TCR sequences that are up- or down-regulated and by assessing by flow cytometry a shift away from TNFR1-expressing T cells and towards TNFR2-expressing T cells.

According to these observations, it is preferable in some embodiments to initiate the manufacturing of T_{REG} cells from input T cells that are enriched for a T_{REG} phenotype on an antigen specificity level, as indicated by shifts in the TNF receptor expression profile and shifts in the TCR repertoire.

### Example 8: Inflammatory Monitoring in ALS Patients: Protein Aggregate Sensitization

In spite of the contention that ALS represents a disease that is partially driven by pathogenic T cells, there exists a paucity of information pertaining to the precise antigens that drive such immune reactions. Potentially, T cell sensitization occurs in response to the protein aggregation that has been well-characterized in the cells of ALS patients. Proteostasis is a complex process that controls intra-cellular protein levels; in various lysosomal storage diseases, insufficient proteostasis results in protein aggregates that can then result in immune sensitization to the aggregates. In a similar manner, we reason that sensitization may occur to protein aggregates found in ALS patients, including SOD-1, TDP-43, and p62.

Based on this information, we will incorporate advanced methods of evaluating protein aggregate sensitization into our assessment of the ALS patient inflammatory state. Specifically, using established methodologies, we will generate a library of potentially immunogenic peptides encompassing the ALS-related protein aggregates, including but not limited to SOD-1, TDP-43, and p62. First, we will utilize these peptides to create a primary in vitro sensitization of normal control T cells to the specific epitopes of the protein aggregates; with the development of this capacity, we will then compare normal control and ALS patients for their de novo capacity to respond to the peptides encompassing the protein aggregates. The development of these assays will be advantageous from several perspectives, including: (1) the frequency and intensity of in vitro reactivity will reflect the level of aggregates in the CNS (will reflect the primary neurodegenerative disease), thereby facilitating diagnostic and risk stratification efforts; and (2) normalization of such reactivity will represent a bio-marker for efficacious therapeutic interventions.

### Example 9: Inflammatory Monitoring in ALS Patients: Motor Neuron Cell Sensitization

As stated above, we hypothesize that T cells in ALS may be directed against the key protein aggregates that have been described for this disease. Alternatively, or additionally, T cells may respond to other CNS antigens that have not been elucidated. Because motor neurons are the primary cell type that undergoes cell death in ALS patients, we hypothesize that antigen-presenting-cells (APC) will process and present motor neuron antigens to T cells for the propagation of ALS disease.

To address this, we will propagate human motor neuron cell lines, expose such cells to molecules like TNF-α that will promote an immunogenic cell death, and then present such cells to patient-specific APC generated through culture of monocytes in IL-4 and GM-CSF to promote dendritic cell differentiation. Subsequently, such APC loaded with motor neuron cell debris will be used in co-culture experiments with autologous T cells from ALS patients or normal controls. After T cell propagation ex vivo, T cells will be tested for enhanced reactivity towards the original motor neuron cell line relative to control cell lines.

An ability to detect and monitor motor neuron-reactive T cells will assist in the diagnosis, risk stratification and therapeutic monitoring of ALS patients.

### Example 10: Inflammatory Monitoring in ALS Patients: Inflammasome Pathway

The NLRP3 inflammasome has been shown to contribute to the pathogenesis of ALS in experimental models. The signaling pathway of the inflammasome has recently been further characterized to involve sequential activation of molecules such as IRF3, IRF7, and MDA5 that ultimately results in the initiation of IL-1β mediated innate inflammation. Because peripheral monocytes are known to traffic through the CNS and acquire inflammatory signals in that micro-environment, circulating monocytes can reflect the tissue disease state. Given this information, we hypothesize that peripheral monocytes in ALS patients will possess an inflammasome signaling motif; in addition, successful therapeutic interventions will reduce this signaling signature.

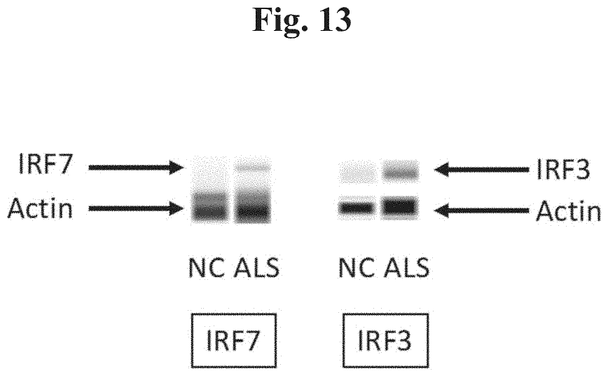

Towards this aim, we have measured the inflammasome signaling cascade using Western Blot analysis. As shown in FIG. 13, peripheral blood mononuclear cells (PBMC) from a normal control (“NC”) and an ALS patient (“ALS”) were harvested, protein was isolated, and western blot was performed to evaluate content of the control gene Actin and two genes in the inflammasome cascade, namely, IRF7 and IRF3. The results demonstrate that ALS patients have evidence of inflammasome activation by western blot analysis of PBMC. As shown in FIG. 13, peripheral blood mononuclear cells from ALS patients can indeed show an increased inflammasome signature.

### Example 11: Inflammatory Monitoring in ALS Patients: Serum Neuronal Molecule Detection

Inflammatory monitoring in ALS patients will be useful as an adjunct to attempts by the field to monitor CNS degeneration through the measurement of CNS molecules in the serum. That is, ALS patients who respond to therapy will have both a normalization of the inflammatory phenotype and normalization of serum markers of CNS degeneration, the specifics of which have been reviewed recently in Beach TG. A Review of Biomarkers for Neurodegenerative Disease: Will They Swing Us Across the Valley? Neurology and Therapy. 2017;6(Suppl 1):5-13. Such markers include but are not limited to: neurofilament light (NF-L); various amyloid-beta-associated peptide fragments; and miRNA species miR-206, 143-3p, and 374b-5p.

### Example 12: Inflammatory Monitoring in ALS Patients: Reduced Cyclic AMP

Adenosine is released during cell death, and is increased in the tissue micro-environment during neurodegenerative diseases such as ALS. Adenosine mediates signals to the immune system via a variety of cell surface adenosine receptors, including the A₁, A_{2A}, A_{2B}, and A₃ receptor types. Adenosine receptors are G protein-coupled receptors that signal at least in part by induction of cyclic AMP, which mediates an immune suppressive effect. During adenosine-driven inflammation, adenosine receptors can be down-regulated in a negative feedback loop. This down-regulation can have negative consequences, as the immune suppressive molecule cyclic AMP might be reduced with adenosine receptor down-regulation.

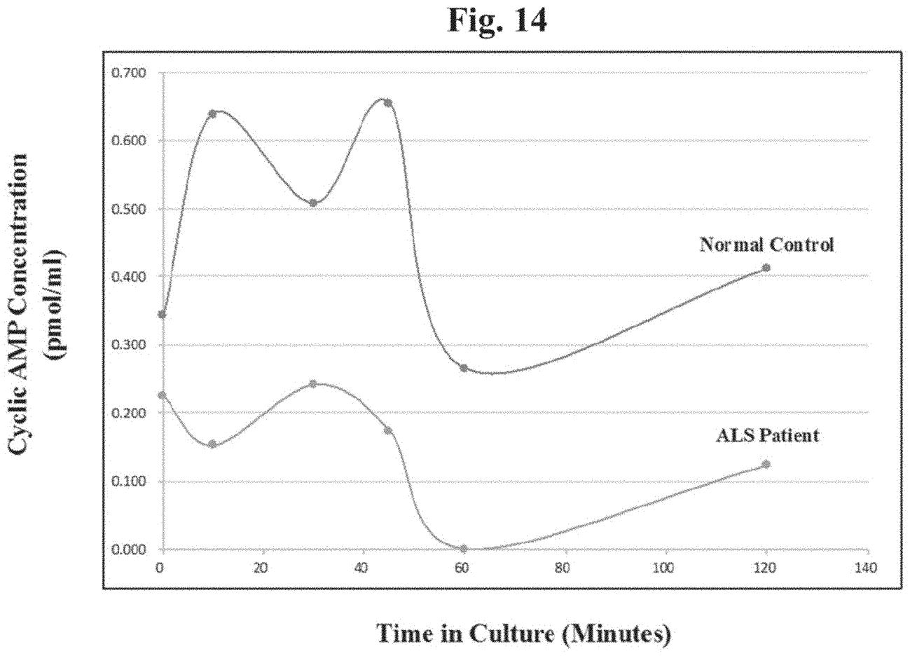

To assess this possibility in ALS patient peripheral blood mononuclear cells, PBMC from an ALS patient and a normal control were isolated and stimulated in culture in the presence of the adenosine A_{2A} receptor agonist CGS21680. The results are shown below in FIG. 14: indeed, ALS patient PBMC have a reduced response to adenosine A_{2A} receptor agonism relative to normal control PBMC. As such, measurement of cyclic AMP under various adenosine receptor stimulation conditions represents a novel approach to diagnose and monitor during therapeutic interventions the inflammatory status of patients with neurodegenerative disease such as ALS.

In some embodiments, measurement of cAMP can be performed on conditioned supernatant after incubation of PBMCs or of cell lysates obtained from cells cultured for a period time. In certain aspects, the conditioned supernatant can comprise a cell lysate. In some embodiments, the measurement of cAMP can be compared to a control value or sample. In some aspects, a 25% reduction in cAMP in the sample after exposure to the agonist or antagonist of the adenosine A₁, A2_{A}, A2_{B} or A₃ receptors is indicative of an inflammatory state. By way of example, but not limitation, a reduction of 25%, 30%, 40%, 50%, 60%, 70%, 80%, 90% or more in the cAMP concentration is indicative of an inflammatory state.

In some embodiments, measurement of cAMP can be combined with measurement of cytokines or other markers of inflammation within the present disclosure to assess the inflammatory state of a subject. By way of example, but not limitation, measurement of at least one cytokine selected from TNF-α, IL-β and IL-6 in the cell lysate and/or supernatant can be used as described in the present disclosure to determine the inflammatory state of the subject.

In the present disclosure, it should be understood that there is no indication that the use of nT_{REGS}, iT_{REGS}, or any combination thereof is to be a treatment option for any disease other than ALS. There is no reason to expect and no understanding that the use of nT_{REGS}, iT_{REGS}, or any combination thereof can be used as a treatment option for any other diseases to which the methods of the present disclosure can be applied. To the extent that the methods of the present disclosure can be used to assess other diseases or conditions, including the inflammatory state of a subject, it should be understood that the use of nT_{REGS}, iT_{REGS}, or any combination thereof as a treatment modality is not contemplated by the present disclosure for those other diseases. Such other diseases can include other neurodegenerative conditions such as age-related macular degeneration (AMD), Parkinson's Disease (PD), Alzheimer's Disease (AD), and Huntington's Disease (HD). The methods of the present disclosure can be modified in such cases. Specifically, by way of example but not limitation, the protein aggregate that will be utilized to assess antigen-specific T cell responses in such conditions may include but not be limited to alpha-synuclein (AMD and PD), tau (AD), and HttExl (HD).

What is claimed is:

1. A method for determining the inflammatory state of a subject suffering from amyotrophic lateral sclerosis (ALS), comprising:
culturing peripheral blood mononuclear cells (PBMCs) comprising CD14⁺ monocytes and CD3⁺ T cells from a subject suffering from ALS in a culture medium supplemented with human serum and CGS21680 or a salt thereof for a period of time to yield a conditioned culture medium comprising a conditioned supernatant;
collecting said conditioned supernatant;
measuring a concentration of at least one cytokine selected from TNF-α, IL-1β and IL-6 in said conditioned supernatant;
comparing said concentration of said at least one cytokine to a concentration of said at least one cytokine in a control sample or a standard value,
wherein an increase in the concentration of the at least one cytokine in the conditioned supernatant relative to the concentration of the at least one cytokine in the control sample or a standard value is indicative that said subject is in an inflammatory state.

2. The method of claim 1, wherein said culture medium further comprises X-Vivo 20 media.

3. The method of claim 1, wherein said culture medium is supplemented with 5% human serum.

4. The method of claim 1, wherein said culture medium contains 0.01 to 10 μM of CGS21680 of the salt thereof.

5. The method of claim 1, wherein said period of time is from 18 to 48 hours.

6. The method of claim 1, wherein said increase in the concentration of the at least one cytokine in the conditioned supernatant relative to the concentration of the at least one cytokine in the control sample is at least a three-fold increase.

7. The method of claim 1, wherein said standard value is 10 pg/mL/1×10⁶ cells/24 hours.

8. The method of claim 1, further comprising, prior to culturing said PBMCs:
isolating said CD14⁺ monocytes and CD3⁺ T cells from a sample comprising said PBMCs from said subject.

9. The method of claim 8, further comprising, prior to isolating said CD14⁺ monocytes and CD3⁺ T cells:
harvesting a sample comprising PBMCs from said subject.

10. The method of claim 1, further comprising, if said subject is in an inflammatory state and said at least one cytokine is TNF-α:
administering an anti-TNF-α therapy.

11. The method of claim 1, further comprising, if said subject is in an inflammatory state and said at least one cytokine is IL-1β:
administering an anti-IL-1β therapy.

12. The method of claim 1, further comprising, if said subject is in an inflammatory state and said at least one cytokine is IL-6:
administering an anti-IL-6 therapy.

13. The method of any one of claims 1-12, further comprising, if said subject is in an inflammatory state:
subjecting said subject to an immune depletion regimen followed by adoptive transfer of manufactured natural T cells (nT_{REGS}), manufactured regulatory T cells (iT_{REGS}), or a combination thereof.

14. A method for determining the inflammatory state of a subject suffering from amyotrophic lateral sclerosis (ALS), comprising:
culturing peripheral blood mononuclear cells (PBMCs) comprising CD14⁺ monocytes and CD3⁺ T cells from a subject suffering from ALS in a culture medium supplemented with human serum and CD40 ligand (CD40L) for a period of time to yield a conditioned culture medium comprising a conditioned supernatant;
collecting said conditioned supernatant;
measuring a concentration of at least one cytokine selected from TNF-α, IL-β and IL-6 in said conditioned supernatant;
comparing said concentration of said at least one cytokine to a concentration of said at least one cytokine in a control sample or a standard value,
wherein an increase in the concentration of the at least one cytokine in the conditioned supernatant relative to the concentration of the at least one cytokine in the control sample or a standard value is indicative that said subject is in an inflammatory state.

15. The method of claim 14, wherein said culture medium comprises X-Vivo 20 media.

16. The method of claim 14, wherein said culture medium is supplemented with 5% human serum.

17. The method of claim 14, wherein said culture medium contains 0.1 to 10 μg/mL CD40L.

18. The method of claim 14, wherein said CD40L is recombinant, human CD40L.

19. The method of claim 14, wherein said period of time is from 18 to 48 hours.

20. The method of claim 14, wherein said increase in the concentration of the at least one cytokine in the conditioned supernatant relative to the concentration of the at least one cytokine in the control sample is at least a three-fold increase.

21. The method of claim 14, wherein said standard value is 10 pg/mL/1×10⁶ cells/24 hours or less.

22. The method of claim 14, further comprising, prior to culturing said PBMCs:
isolating said CD14⁺ monocytes and CD3⁺ T cells from a sample comprising said PBMCs from said subject.

23. The method of claim 22, further comprising, prior to isolating said CD14⁺ monocytes and CD3⁺ T cells:
harvesting a sample comprising PBMCs from said subject.

24. The method of claim 14, further comprising, if said subject is in an inflammatory state and said at least one cytokine is TNF-α:
administering an anti-TNF-α therapy.

25. The method of claim 14, further comprising, if said subject is in an inflammatory state and said at least one cytokine is IL-β:
administering an anti-IL-β therapy, either alone or in combination with an anti-TNF-α therapy.

26. The method of claim 14, further comprising, if said subject is in an inflammatory state and said at least one cytokine is IL-6:
administering an anti-IL-6 therapy, either alone or in combination with an anti-TNF-α therapy.

27. The method of any one of claims 14-26, further comprising, if said subject is in an inflammatory state:
subjecting said subject to an immune depletion regimen followed by adoptive transfer of natural T cells (nT_{REGS}), manufactured regulatory T cells (iT_{REGS}), or a combination thereof.

28. A method for determining the inflammatory state of a subject suffering from amyotrophic lateral sclerosis (ALS), comprising:
performing flow cytometry of a population of PBMCs in a sample from a subject suffering from ALS to determine a percentage of CD4⁺ or CD8⁺ T cells in said population of PBMCs that co-express FoxP3 and a marker selected from T-bet, IL-2 and IFN-γ;
comparing the percentages of CD4⁺ and CD8⁺ T cells in said population of PBMCs that co-express FoxP3 and said marker to a percentage of CD4⁺ and CD8⁺ T cells that co-express FoxP3 and said marker in a control sample or to a standard value for CD4⁺ and CD8⁺ T cells,
wherein an increase in the percentage of CD4⁺ and CD8⁺ T cells that co-express FoxP3 and said marker relative to the percentage of CD4⁺ and CD8⁺ T cells in a control sample or a standard value for CD4⁺ and CD8⁺ T cells is indicative that said subject is in an inflammatory state.

29. The method of claim 28, wherein said step of performing flow cytometry comprises:
adding a permeabilization reagent to said population of PBMCs;
adding to a said population of PBMCs:
a) a first labeling molecule comprising a first binding domain capable of specifically binding CD4 and a first label,
b) a second labeling molecule comprising a second binding domain capable of specifically binding CD8 and a second label,
c) a third labeling molecule comprising a third binding domain capable of specifically binding FoxP3 and a third label, and
d) a fourth labeling molecule comprising a fourth binding domain capable of specifically binding a marker selected from T-bet, IL-2 and IFN-γ and a fourth label;
incubating said population of PBMCs and said first labeling molecule, said second labeling molecule, said third labeling molecule and said fourth labeling molecule under conditions sufficient for said first binding domain to bind CD4, said second binding domain to bind CD8, said third binding domain to bind FoxP3, and said fourth binding domain to bind said marker to yield a labeled population of PBMCs;
passing said labeled population of PBMCs through a flow cytometer configured to count cells based on said first label, second label, third label, and fourth label to determine a first number of cells bound to said first labeling molecule, a second number of cells bound to said first labeling molecule, said third labeling molecule and said fourth labeling molecule, a third number of cells bound to said second labeling molecule, and a fourth number of cells bound to said second labeling molecule, said third labeling molecule, and said fourth labeling molecule;
calculating a percentage of CD4⁺ T cells in said population of PBMCs that co-express FoxP3 and said marker by calculating said second number as a percentage of said first number;
calculating a percentage of CD8⁺ T cells in said population of PBMCs that co-express FoxP3 and said marker by calculating said fourth number as a percentage of said third number

30. The method of any one of claims 28-29, wherein said first binding domain is an anti-CD4 antibody or fragment thereof.

31. The method of any one of claims 28-30, wherein said second binding domain is an anti-CD8 antibody or fragment thereof.

32. The method of any one of claims 28-31, wherein said third binding domain is an anti-FoxP3 antibody or fragment thereof.

33. The method of any one of claims 28-32, wherein said marker is T-bet, and wherein said fourth binding domain is an anti-T-bet antibody or fragment thereof.

34. The method of any one of claims 28-32, wherein said marker is IL-2, and wherein said fourth binding domain is an anti-IL-2 antibody or fragment thereof.

35. The method of any one of claims 28-32, wherein said marker is IFN-γ, and wherein said fourth binding domain is an anti-IFN-γ antibody or fragment thereof.

36. The method of any one of claims 28-35, wherein said standard value is less than 10%.

37. The method of any one of claims 28-36, wherein said increase in the percentage of CD4⁺ and CD8⁺ T cells is at least three times higher than the standard value.

38. The method of any one of claims 28-36, wherein said increase in the percentage of CD4⁺ and CD8⁺ T cells is at least three times higher than the control sample.

39. The method of any one of claims 28-38, further comprising:
harvesting a sample comprising said population of PBMCs from said subject.

40. The method of any one of claims 28-39, further comprising, when said subject is in an inflammatory state:
administering to said subject natural T cells (nT_{REGS}), manufactured regulatory T cells (iT_{REGS}), or a combination thereof.

41. The method of claim 40, further comprising, prior to administering to said subject natural T cells (nT_{REGS}), manufactured regulatory T cells (iT_{REGS}), or a combination thereof:
subjecting said subject to an immune depletion regimen to reduce at least a portion of CD4⁺ Th1 and CD8⁺ Tc1 cells in said subject.

42. A method for determining the inflammatory state of a subject suffering from amyotrophic lateral sclerosis (ALS), comprising:
performing flow cytometry of a population of peripheral blood mononuclear cells (PBMCs) in a sample from a subject suffering from ALS to determine a percentage of monocytes in said population of PBMCs that co-express CD16 and CD86 out of the total number of CD16⁺ monocytes, a percentage of monocytes in said population of PBMCs that co-express CD16 and CD80 out of the total number of CD16⁺ monocytes, a percentage of monocytes in said population of PBMCs that co-express CD14 and CD86 out of the total number of CD14⁺ monocytes, and a percentage of monocytes in said population of PBMCs that co-express CD14 and CD80 out of the total number of CD14⁺ monocytes;
comparing the percentage of monocytes in said population of PBMCs that co-express CD16 and CD86 out of the total number of CD16⁺ monocytes to a percentage of monocytes that co-express CD16 and CD86 out of the total number of CD16⁺ monocytes in a control sample or to a standard value for CD16⁺ monocytes,
comparing the percentage of monocytes in said population of PBMCs that co-express CD16 and CD80 out of the total number of CD16⁺ monocytes to a percentage of monocytes that co-express CD16 and CD80 out of the total number of CD16⁺ monocytes in a control sample or to a standard value for CD16⁺ monocytes
comparing the percentage of monocytes in said population of PBMCs that co-express CD14 and CD86 out of the total number of CD14⁺ monocytes to a percentage of monocytes that co-express CD14 and CD86 out of the total number of CD14⁺ monocytes in a control sample or to a standard value for CD14⁺ monocytes
comparing the percentage of monocytes in said population of PBMCs that co-express CD14 and CD80 out of the total number of CD14⁺ monocytes to a percentage of monocytes that co-express CD14 and CD80 out of the total number of CD14⁺ monocytes in a control sample or to a standard value for CD14⁺ monocytes,
wherein an increase in the percentage of monocytes in said population of PBMCs that co-express CD16 and CD86 out of the total number of CD16⁺ monocytes in the population of PBMCs relative to the percentage of monocytes that co-express CD16 and CD86 out of the total number of CD16⁺ monocytes in the control sample or to the standard value for CD16⁺ monocytes is indicative that said subject is in an inflammatory state,
wherein an increase in the percentage of monocytes in said population of PBMCs that co-express CD16 and CD80 out of the total number of CD16⁺ monocytes in the population of PBMCs relative to the percentage of monocytes that co-express CD16 and CD80 out of the total number of CD16⁺ monocytes in the control sample or to the standard value for CD16⁺ monocytes is indicative that said subject is in an inflammatory state
wherein an increase in the percentage of monocytes in said population of PBMCs that co-express CD14 and CD86 out of the total number of CD14⁺ monocytes in the population of PBMCs relative to the percentage of monocytes that co-express CD14 and CD86 out of the total number of CD14⁺ monocytes in the control sample or to the standard value for CD14⁺ monocytes is indicative that said subject is in an inflammatory state
and wherein an increase in the percentage of monocytes in said population of PBMCs that co-express CD14 and CD80 out of the total number of CD14⁺ monocytes in the population of PBMCs relative to the percentage of monocytes that co-express CD14 and CD80 out of the total number of CD14⁺ monocytes in the control sample or to the standard value for CD14⁺ monocytes is indicative that said subject is in an inflammatory state.

43. The method of claim 42, further comprising:
harvesting a sample comprising said population of PBMCs from said subject.

44. The method of any one of claims 42-43, wherein said step of performing flow cytometry of said population of PBMCs comprises:
adding to said population of PBMCs:
a) a first labeling molecule comprising a first binding domain capable of specifically binding CD16 and a first label,
b) a second labeling molecule comprising a second binding domain capable of specifically binding CD14 and a second label,
c) a third labeling molecule comprising a third binding domain capable of specifically binding CD86 and a third label, and
d) a fourth labeling molecule comprising a fourth binding domain capable of specifically binding CD80 and a fourth label;
incubating said population of PBMCs and said first labeling molecule, said second labeling molecule, said third labeling molecule, and said fourth labeling molecule under conditions sufficient for said first binding domain to bind CD16, said second binding domain to bind CD14, said third binding domain to bind CD86, and said fourth binding domain to bind CD80 to yield a labeled population of PBMCs;
passing said labeled population of PBMCs through a flow cytometer configured to count cells based on said first label, second label, said third label, and said fourth label to determine a first number of cells bound to said first label, a second number of cells bound to said first label and said third label, a third number of cells bound to said first label and said fourth label, a fourth number of cells bound to said second label, a fifth number of cells bound to said second label and said third label, and a sixth number of cells bound to said second label and said fourth label;
calculating said percentage of monocytes in said population of PBMCs that co-express CD16 and CD86 out of the total number of CD16⁺ monocytes by calculating said second number as a percentage of said first number;
calculating said percentage of monocytes in said population of PBMCs that co-express CD16 and CD80 out of the total number of CD16⁺ monocytes by calculating said third number as a percentage of said first number;
calculating said percentage of monocytes in said population of PBMCs that co-express CD14 and CD86 out of the total number of CD14⁺ monocytes by calculating said fifth number as a percentage of said fourth number;
calculating said percentage of monocytes in said population of PBMCs that co-express CD14 and CD80 out of the total number of CD14⁺ monocytes by calculating said sixth number as a percentage of said fourth number.

45. The method of any one of claims 42-44, wherein said first binding domain is an anti-CD16 antibody or fragment thereof.

46. The method of any one of claims 42-45, wherein said second binding domain is an anti-CD86 antibody or fragment thereof.

47. The method of any one of claims 42-46, wherein said standard value for CD16⁺ monocytes is less than 10%.

48. The method of any one of claims 42-46, wherein said increase in the percentage of monocytes in said population of PBMCs that co-express CD16 and CD86 out of the total number of CD16⁺ monocytes in the population of PBMCs relative to the percentage of monocytes that co-express CD16 and CD86 out of the total number of CD16⁺ monocytes in the control sample is an increase of from at least three-fold the percentage of CD16⁺ monocytes that co-express CD16 and CD86 out of the total number of CD16⁺ monocytes in the control sample.

49. The method of any one of claims 42-46, wherein said increase in the percentage of monocytes in said population of PBMCs that co-express CD16 and CD86 out of the total number of CD16⁺ monocytes in the population of PBMCs relative to the standard value for CD16⁺ monocytes is an increase of at least three-fold the standard value for CD16⁺ monocytes.

50. The method of any one of claims 42-49, further comprising, when said subject is in an inflammatory state:
administering to said subject a composition comprising natural T cells (nT_{REGS}), manufactured regulatory T cells (iT_{REGS}), or a combination thereof.

51. The method of claim 50, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

52. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS comprising CD14⁺ monocytes and CD3⁺ T cells in a culture medium supplemented with human serum for a period of time to yield a conditioned culture medium comprising a conditioned supernatant;
collecting said conditioned supernatant;
measuring a concentration of at least one cytokine selected from TNF-α, IL-β and IL-6 in said conditioned supernatant;
comparing said concentration of the at least one cytokine selected from TNF-α, IL-β and IL-6 to a concentration of said at least one cytokine in a control sample or a standard value,
wherein an increase in the concentration of the at least one cytokine in the conditioned supernatant relative to the concentration of the at least one cytokine in the control sample or standard value is indicative that said subject is in an inflammatory state.

53. The method of claim 52, wherein said culture medium is supplemented with 5% human serum.

54. The method of claim 52, further comprising:
harvesting said sample comprising said population of PBMCs from said subject.

55. The method of claim 52, wherein said period of time is from about 16 to about 48 hours.

56. The method of claim 52, wherein said period of time is 24 hours.

57. The method of claim 52, further comprising, prior to culturing said at least a portion of said PBMCs:
isolating CD14⁺ monocytes and CD3⁺ T cells from said population of PBMCs to yield said at least a portion of said PBMCs.

58. The method of claim 52, wherein said increase in the concentration of the at least on cytokine in the conditioned supernatant relative to the concentration of the at least one cytokine in the control sample is at least an increase of at least three times the concentration of the at least one cytokine in the control sample.

59. The method of claim 52, wherein said standard value is less than 10 pg/mL/le6 cells/24 hours.

60. The method of claim 52, further comprising, if said subject is in an inflammatory state and said at least one cytokine is TNF-α:
administering to said subject an anti-TNF-α therapy.

61. The method of claim 52, further comprising, if said subject is in an inflammatory state and said at least one cytokine is IL-β:
administering to said subject an anti-IL-β therapy, either alone or in combination with an anti-TNF-α therapy..

62. The method of claim 52, further comprising, if said subject is in an inflammatory state and said at least one cytokine is IL-6:
administering to said subject an anti-IL-6 therapy, either alone or in combination with an anti-TNF-α therapy..

63. The method of any one of claims 52-62, further comprising, if said subject is in an inflammatory state:
administering to said subject a composition comprising natural T cells (nT_{REGS}), manufactured regulatory T cells (iT_{REGS}), or a combination thereof.

64. The method of claim 63, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

65. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS and being treated with an anti-TNF-α therapy comprising CD14⁺ monocytes and CD3⁺ T cells in a culture medium supplemented with human serum for a period of time to yield a conditioned culture medium comprising a conditioned supernatant;
collecting said conditioned supernatant;
measuring a concentration of TNF-α in said conditioned supernatant;
comparing said concentration of TNF-α to a concentration of TNF-α in a control sample or a standard value,
wherein an increase in the concentration of TNF-α in the conditioned supernatant relative to the concentration of TNF-α in the control sample or standard value is indicative that said subject is in an inflammatory state.

66. The method of claim 65, wherein said culture medium is supplemented with 5% human serum.

67. The method of any one of claims 65-66, further comprising:
harvesting said sample comprising said population of PBMCs from said subject.

68. The method of any one of claims 65-67, wherein said period of time is from about 16 hours to about 48 hours.

69. The method of any one of claims 65-67, wherein said period of time is 24 hours.

70. The method of any one of claims 65-69, further comprising, prior to culturing said at least a portion of said PBMCs:
isolating CD14⁺ monocytes and CD3⁺ T cells from said population of PBMCs to yield said at least a portion of said PBMCs.

71. The method of any one of claims 65-70, wherein said increase in the concentration of TNF-α in the conditioned supernatant relative to the concentration of TNF-α in the control sample is at least an increase of at least three times the concentration of TNF-α in the control sample.

72. The method of any one of claims 65-70, wherein said standard value is less than 10 pg/mL/1×10⁶ cells/24 hours.

73. The method of any one of claims 65-72, further comprising, if said subject is in an inflammatory state:
adjusting said anti-TNF-α therapy by increasing a dosage or duration of treatment of said anti-TNF-α therapy.

74. The method of any one of claims 65-73, wherein said anti-TNF-α therapy comprises administering etanercept to said subject.

75. A method for determining the inflammatory state of a subject suffering from amyotrophic lateral sclerosis (ALS), comprising:
culturing a first portion of peripheral blood mononuclear cells (PBMCs) from sample comprising a population of PBMCs from a subject suffering from ALS comprising CD14⁺ monocytes and CD3⁺ T cells in a first culture medium supplemented with human serum and CD40 ligand (CD40L) for a period of time to yield a first conditioned culture medium comprising a first conditioned supernatant;
culturing a second portion of said PBMCs from said sample comprising CD14⁺ monocytes and CD3⁺ T cells in a second culture medium supplemented with human serum for a period of time to yield a second conditioned culture medium comprising a second conditioned supernatant;
collecting said first conditioned supernatant and said second conditioned supernatant;
measuring a concentration of at least one cytokine selected from TNF-α, IL-β and IL-6 in said first conditioned supernatant and said second conditioned supernatant;
comparing said concentration of the at least one cytokine selected from TNF-α, IL-β and IL-6 in said first conditioned supernatant to said concentration of said at least one cytokine in said second conditioned supernatant,
wherein an increase in the concentration of the at least one cytokine in the first conditioned supernatant relative to the concentration of the at least one cytokine in the second conditioned supernatant is indicative that said subject is in an inflammatory state.

76. The method of claim 75, further comprising:
harvesting said sample comprising said population of PBMCs from said subject.

77. The method of any one of claims 75-76, wherein said CD40L is added to said culture medium at a concentration of 0.1 to 10 μg/mL.

78. The method of any one of claims 75-76, wherein said CD40L is added to said culture medium at a concentration of 1.0 μg/mL.

79. The method of any one of claims 75-78, wherein said CD40L is recombinant, human CD40L.

80. The method of any one of claims 75-79, wherein said period of time is from about 18 to about 48 hours.

81. The method of any one of claims 75-79, wherein said period of time is 24 hours.

82. The method of any one of claims 75-81, wherein said first culture medium and said second culture medium are supplemented with 5% human serum.

83. The method of any one of claims 75-82, further comprising, prior to culturing said at least a portion of said PBMCs:
isolating CD14⁺ monocytes and CD3⁺ T cells from said population of PBMCs to yield said first portion of said PBMCs and said second portion of said PBMCs.

84. The method of any one of claims 75-83, wherein said increase in the concentration of the at least on cytokine in the first conditioned supernatant relative to the concentration of the at least one cytokine in the second conditioned supernatant is at least an increase of at least three times the concentration of the at least one cytokine in the second conditioned supernatant.

85. The method of any one of claims 75-84, further comprising, if said subject is in an inflammatory state and said at least one cytokine is TNF-α:
administering to said subject an anti-TNF-α therapy.

86. The method of any one of claims 75-84, further comprising, if said subject is in an inflammatory state and said at least one cytokine is IL-β:
administering to said subject an anti-IL-β therapy, either alone or in combination with an anti-TNF-α therapy.

87. The method of any one of claims 75-84, further comprising, if said subject is in an inflammatory state and said at least one cytokine is IL-6:
administering to said subject an anti-IL-6 therapy, either alone or in combination with an anti-TNF-α therapy.

88. The method of any one of claims 75-84, further comprising, if said subject is in an inflammatory state:
administering to said subject a targeted pharmacologic inhibitor of TRAF6.

89. The method of any one of claims 75-88, further comprising, if said subject is in an inflammatory state:
administering to said subject a composition comprising natural T cells (nT_{REGS}), manufactured regulatory T cells (iT_{REGS}), or a combination thereof.

90. The method of claim 89, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

91. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS comprising CD14⁺ monocytes and CD3⁺ T cells in a culture medium supplemented with human serum and an agonist or antagonist of the adenosine A₁, A_{2A}, A₂B or A₃ receptors for a period of time to yield a conditioned culture medium comprising a conditioned supernatant;
collecting said conditioned supernatant;
measuring a concentration of at least one cytokine selected from TNF-α, IL-β and IL-6 in said conditioned supernatant;
comparing said concentration of the at least one cytokine selected from TNF-α, IL-β and IL-6 to a concentration of said at least one cytokine in a control sample or a standard value,
wherein an increase in the concentration of the at least one cytokine in the conditioned supernatant relative to the concentration of the at least one cytokine in the control sample or standard value is indicative that said subject is in an inflammatory state.

92. The method of claim 91, further comprising:
harvesting said sample comprising said population of PBMCs from said subject.

93. The method of any one of claims 91-92, wherein said period of time is from about 18 to about 48 hours.

94. The method of any one of claims 91-92, wherein said period of time is 24 hours.

95. The method of any one of claims 91-94, wherein said agonist or antagonist of the adenosine A₁, A_{2A}, A₂B or A₃ receptors is present said culture medium at a concentration between 0.01 to 10 μM.

96. The method of any one of claims 91-94, wherein said agonist or antagonist of the adenosine A₁, A_{2A}, A₂B or A₃ receptors is present said culture medium at a concentration of 1 μM.

97. The method of any one of claims 91-96, further comprising, prior to culturing said at least a portion of said PBMCs:
isolating CD14⁺ monocytes and CD3⁺ T cells from said population of PBMCs to yield said at least a portion of said PBMCs.

98. The method of claim 97, wherein said at least portion of said PBMCs comprises CD14⁺ monocytes without CD3⁺ T cells.

99. The method of claim 97, wherein said at least a portion of said PBMCs comprises CD3⁺ T cells without CD14⁺ monocytes.

100. The method of any one of claims 91-99, wherein said increase in the concentration of the at least on cytokine in the conditioned supernatant relative to the concentration of the at least one cytokine in the control sample is at least an increase of at least three times the concentration of the at least one cytokine in the control sample.

101. The method of any one of claims 91-99, wherein said standard value is less than 10 pg/mL/1×10⁶ cells/24 hours.

102. The method of any one of claims 91-101, wherein said culture medium is supplemented with 5% human serum.

103. The method of any one of claims 91-102, further comprising, if said subject is in an inflammatory state and said at least one cytokine is TNF-α:
administering to said subject an anti-TNF-α therapy.

104. The method of any one of claims 91-102, further comprising, if said subject is in an inflammatory state and said at least one cytokine is IL-β:
administering to said subject an anti-IL-β therapy, either alone or in combination with an anti-TNF-α therapy.

105. The method of any one of claims 91-102, further comprising, if said subject is in an inflammatory state and said at least one cytokine is IL-6:
administering to said subject an anti-IL-6 therapy, either alone or in combination with an anti-TNF-α therapy.

106. The method of any one of claims 91-105, further comprising, if said subject is in an inflammatory state:
administering to said subject a composition comprising natural T cells (nT_{REGS}), manufactured regulatory T cells (iT_{REGS}), or a combination thereof.

107. The method of claim 106, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

108. A method for determining the inflammatory state of a subject suffering from amyotrophic lateral sclerosis (ALS), comprising:
culturing a first portion of peripheral blood mononuclear cells (PBMCs) from a sample comprising a population of PBMCs from a subject suffering from ALS comprising CD14⁺ monocytes and CD3⁺ T cells in a first culture medium supplemented with human serum and an agonist or antagonist of the adenosine A₁, A_{2A}, A₂B or A₃ receptors at a concentration of about 0.01 to 10 μM for a period of time to yield a first conditioned culture medium comprising a first conditioned supernatant;
culturing a second portion of said PBMCs from said sample comprising CD14⁺ monocytes and CD3⁺ T cells in a second culture medium supplemented with human serum for a period of time to yield a second conditioned culture medium comprising a second conditioned supernatant;
collecting said first conditioned supernatant and said second conditioned supernatant;
measuring a concentration of at least one cytokine selected from TNF-α, IL-β and IL-6 in said first conditioned supernatant and said second conditioned supernatant;
comparing said concentration of the at least one cytokine selected from TNF-α, IL-β and IL-6 in said first conditioned supernatant to said concentration of said at least one cytokine in said second conditioned supernatant,
wherein an increase in the concentration of the at least one cytokine in the first conditioned supernatant relative to the concentration of the at least one cytokine in the second conditioned supernatant is indicative that said subject is in an inflammatory state.

109. The method of claim 108, further comprising:
harvesting said sample comprising said population of PBMCs from said subject.

110. The method of any one of claims 108-109, wherein said first culture medium and said second culture medium are supplemented with 5% human serum.

111. The method of any one of claims 108-110, wherein said period of time is from about 18 to about 48 hours.

112. The method of any one of claims 108-110, wherein said period of time is 24 hours.

113. The method of any one of claims 108-112, further comprising, prior to culturing said at least a portion of said PBMCs:
isolating CD14⁺ monocytes and CD3⁺ T cells from said population of PBMCs to yield said first portion of said PBMCs and said second portion of said PBMCs.

114. The method of any one of claims 108-113, wherein said increase in the concentration of the at least on cytokine in the first conditioned supernatant relative to the concentration of the at least one cytokine in the second conditioned supernatant is at least an increase of at least three times the concentration of the at least one cytokine in the second conditioned supernatant.

115. The method of any one of claims 108-114, further comprising, if said subject is in an inflammatory state and said at least one cytokine is TNF-α:
administering to said subject an anti-TNF-α therapy.

116. The method of any one of claims 108-114, further comprising, if said subject is in an inflammatory state and said at least one cytokine is IL-β:
administering to said subject an anti-IL-β therapy, either alone or in combination with an anti-TNF-α therapy.

117. The method of any one of claims 108-114, further comprising, if said subject is in an inflammatory state and said at least one cytokine is IL-6:
administering to said subject an anti-IL-6 therapy, either alone or in combination with an anti-TNF-α therapy.

118. The method of any one of claims 108-117, further comprising, if said subject is in an inflammatory state:
administering to said subject a composition comprising natural T cells (nT_{REGS}), manufactured regulatory T cells (iT_{REGS}), or a combination thereof.

119. The method of claim 118, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

120. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS comprising CD14⁺ monocytes and CD3⁺ T cells in a culture medium supplemented with human serum and a T cell checkpoint inhibitor at a concentration of 0.001 to 10 μg/mL for a period of time to yield a conditioned culture medium comprising a conditioned supernatant;
collecting said conditioned supernatant;
measuring a concentration of at least one cytokine selected from TNF-α, IL-β and IL-6 in said conditioned supernatant;
comparing said concentration of the at least one cytokine selected from TNF-α, IL-β and IL-6 to a concentration of said at least one cytokine in a control sample or a standard value,
wherein an increase in the concentration of the at least one cytokine in the conditioned supernatant relative to the concentration of the at least one cytokine in the control sample or standard value is indicative that said subject is in an inflammatory state.

121. The method of claim 120, wherein said culture medium is supplemented with 5% human serum.

122. The method of any one of claims 120-121, further comprising:
harvesting said sample comprising said population of PBMCs from said subject.

123. The method of claim 120, wherein said period of time is from about 18 hours to about 48 hours.

124. The method of claim 120, wherein said period of time is 24 hours.

125. The method of claim 120, further comprising, prior to culturing said at least a portion of said PBMCs:
isolating CD14⁺ monocytes and CD3⁺ T cells from said population of PBMCs to yield said at least a portion of said PBMCs.

126. The method of any one of claims 120-125, wherein said increase in the concentration of the at least on cytokine in the conditioned supernatant relative to the concentration of the at least one cytokine in the control sample is at least an increase of at least three times the concentration of the at least one cytokine in the control sample.

127. The method of claim 120, wherein said standard value is less than 10 pg/mL/1×10⁶ cells/24 hours.

128. The method of claim 120, wherein said T cell checkpoint inhibitor is an anti-PDL1 or anti-PD1 antibody or fragment thereof.

129. The method of claim 120, wherein said T cell checkpoint inhibitor is an anti-CTLA4 antibody or fragment thereof.

130. The method of claim 120, wherein said T cell checkpoint inhibitor is an anti-TIM3 antibody or fragment thereof.

131. The method of claim 120, further comprising, if said subject is in an inflammatory state and said at least one cytokine is TNF-α:
administering to said subject an anti-TNF-α therapy.

132. The method of claim 120, further comprising, if said subject is in an inflammatory state and said at least one cytokine is IL-β:
administering to said subject an anti-IL-β therapy, either alone or in combination with an anti-TNF-α therapy.

133. The method of claim 120, further comprising, if said subject is in an inflammatory state and said at least one cytokine is IL-6:
administering to said subject an anti-IL-6 therapy, either alone or in combination with an anti-TNF-α therapy.

134. The method of any one of claims 120-133, further comprising, if said subject is in an inflammatory state:
administering to said subject a composition comprising natural T cells (nT_{REGS}), manufactured regulatory T cells (iT_{REGS}), or a combination thereof.

135. The method of claim 134, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

136. A method for determining the inflammatory state of a subject suffering from amyotrophic lateral sclerosis (ALS), comprising:
culturing a first portion of peripheral blood mononuclear cells (PBMCs) from a sample comprising a population of PBMCs from a subject suffering from ALS comprising CD14⁺ monocytes and CD3⁺ T cells in a first culture medium supplemented with human serum and a T cell checkpoint inhibitor at a concentration of 0.001 to 10 μg/mL for a period of time to yield a first conditioned culture medium comprising a first conditioned supernatant;
culturing a second portion of said PBMCs from said sample comprising CD14⁺ monocytes and CD3⁺ T cells in a second culture medium supplemented with human serum for a period of time to yield a second conditioned culture medium comprising a second conditioned supernatant;
collecting said first conditioned supernatant and said second conditioned supernatant;
measuring a concentration of at least one cytokine selected from TNF-α, IL-β and IL-6 in said first conditioned supernatant and said second conditioned supernatant;
comparing said concentration of the at least one cytokine selected from TNF-α, IL-β and IL-6 in said first conditioned supernatant to said concentration of said at least one cytokine in said second conditioned supernatant,
wherein an increase in the concentration of the at least one cytokine in the first conditioned supernatant relative to the concentration of the at least one cytokine in the second conditioned supernatant is indicative that said subject is in an inflammatory state.

137. The method of claim 136, further comprising:
harvesting said sample comprising said population of PBMCs from said subject.

138. The method of any one of claims 136-137, wherein said first culture medium and said second culture medium are supplemented with 5% human serum.

139. The method of any one of claims 136-138, wherein said period of time is from about 18 to about 48 hours.

140. The method of any one of claims 136-138, wherein said period of time is 24 hours.

141. The method of any one of claims 136-140, further comprising, prior to culturing said at least a portion of said PBMCs:
isolating CD14⁺ monocytes and CD3⁺ T cells from said population of PBMCs to yield said first portion of said PBMCs and said second portion of said PBMCs.

142. The method of any one of claims 136-141, wherein said increase in the concentration of the at least on cytokine in the first conditioned supernatant relative to the concentration of the at least one cytokine in the second conditioned supernatant is at least an increase of at least three times the concentration of the at least one cytokine in the second conditioned supernatant.

143. The method of any one of claims 136-142, wherein said T cell checkpoint inhibitor is an anti-PDL1 or anti-PD1 antibody or fragment thereof.

144. The method of any one of claims 136-142, wherein said T cell checkpoint inhibitor is an anti-CTLA4 antibody or fragment thereof.

145. The method of any one of claims 136-142, wherein said T cell checkpoint inhibitor is an anti-TIM3 antibody or fragment thereof.

146. The method of any one of claims 136-145, further comprising, if said subject is in an inflammatory state and said at least one cytokine is TNF-α:
administering to said subject an anti-TNF-α therapy.

147. The method of any one of claims 136-145, further comprising, if said subject is in an inflammatory state and said at least one cytokine is IL-β:
administering to said subject an anti-IL-β therapy, either alone or in combination with an anti-TNF-α therapy.

148. The method of any one of claims 136-145, further comprising, if said subject is in an inflammatory state and said at least one cytokine is IL-6:
administering to said subject an anti-IL-6 therapy, either alone or in combination with an anti-TNF-α therapy.

149. The method of any one of claims 136-148, further comprising, if said subject is in an inflammatory state:
administering to said subject a composition comprising natural T cells (nT_{REGS}), manufactured regulatory T cells (iT_{REGS}), or a combination thereof.

150. The method of claim 149, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

151. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS comprising CD14⁺ monocytes and CD3⁺ T cells in a culture medium supplemented with human serum and a monocyte checkpoint inhibitor for a period of time to yield a conditioned culture medium comprising a conditioned supernatant;
collecting said conditioned supernatant;
measuring a concentration of at least one cytokine selected from TNF-α, IL-β and IL-6 in said conditioned supernatant;
comparing said concentration of the at least one cytokine selected from TNF-α, IL-β and IL-6 to a concentration of said at least one cytokine in a control sample or a standard value,
wherein an increase in the concentration of the at least one cytokine in the conditioned supernatant relative to the concentration of the at least one cytokine in the control sample or standard value is indicative that said subject is in an inflammatory state.

152. The method of claim 151, further comprising:
harvesting said sample comprising said population of PBMCs from said subject.

153. The method of any one of claims 151-152, wherein said culture medium is supplemented with 5% human serum.

154. The method of any one of claims 151-153, wherein said period of time is from about 18 hours to about 48 hours.

155. The method of any one of claims 151-154, wherein said period of time is 24 hours.

156. The method of any one of claims 151-155, wherein said monocyte checkpoint inhibitor is present in said culture medium at a concentration of 0.01 to 10 μg/mL.

157. The method of any one of claims 151-155, wherein said monocyte checkpoint inhibitor is present in said culture medium at a concentration of 1 μg/mL.

158. The method of any one of claims 151-157, further comprising, prior to culturing said at least a portion of said PBMCs:
isolating CD14⁺ monocytes and CD3⁺ T cells from said population of PBMCs to yield said at least a portion of said PBMCs.

159. The method of any one of claims 151-158, wherein said increase in the concentration of the at least on cytokine in the conditioned supernatant relative to the concentration of the at least one cytokine in the control sample is at least an increase of at least three the concentration of the at least one cytokine in the control sample.

160. The method of any one of claims 151-159, wherein said standard value is 10 pg/mL/1×10⁶ cells/24 hours.

161. The method of any one of claims 151-160, wherein said monocyte checkpoint inhibitor is an anti-CD200L or anti-CD200 antibody or fragment thereof.

162. The method of any one of claims 151-161, further comprising, if said subject is in an inflammatory state and said at least one cytokine is TNF-α:
administering to said subject an anti-TNF-α therapy.

163. The method of any one of claims 151-161, further comprising, if said subject is in an inflammatory state and said at least one cytokine is IL-β:
administering to said subject an anti-IL-β therapy, either alone or in combination with an anti-TNF-α therapy.

164. The method of any one of claims 151-161, further comprising, if said subject is in an inflammatory state and said at least one cytokine is IL-6:
administering to said subject an anti-IL-6 therapy, either alone or in combination with an anti-TNF-α therapy.

165. The method of any one of claims 151-164, further comprising, if said subject is in an inflammatory state:
administering to said subject a composition comprising natural T cells (nT_{REGS}), manufactured regulatory T cells (iT_{REGS}), or a combination thereof.

166. The method of claim 165, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

167. A method for determining the inflammatory state of a subject suffering from amyotrophic lateral sclerosis (ALS), comprising:
culturing a first portion of peripheral blood mononuclear cells (PBMCs) from a sample comprising a population of PBMCs from a subject suffering from ALS comprising CD14⁺ monocytes and CD3⁺ T cells in a first culture medium supplemented with human serum and a monocyte checkpoint inhibitor for a period of time to yield a first conditioned culture medium comprising a first conditioned supernatant;
culturing a second portion of said PBMCs from said sample comprising CD14⁺ monocytes and CD3⁺ T cells in a second culture medium supplemented with human serum for a period of time to yield a second conditioned culture medium comprising a second conditioned supernatant;
collecting said first conditioned supernatant and said second conditioned supernatant;
measuring a concentration of at least one cytokine selected from TNF-α, IL-β and IL-6 in said first conditioned supernatant and said second conditioned supernatant;
comparing said concentration of the at least one cytokine selected from TNF-α, IL-β and IL-6 in said first conditioned supernatant to said concentration of said at least one cytokine in said second conditioned supernatant,
wherein an increase in the concentration of the at least one cytokine in the first conditioned supernatant relative to the concentration of the at least one cytokine in the second conditioned supernatant is indicative that said subject is in an inflammatory state.

168. The method of claim 167, further comprising:
harvesting said sample comprising said population of PBMCs from said subject.

169. The method of any one of claims 167-168, wherein said first culture medium and said second culture medium are supplemented with 5% human serum.

170. The method of any one of claims 167-169, wherein said period of time is from about 18 hours to about 48 hours.

171. The method of any one of claims 167-169, wherein said period of time is 24 hours.

172. The method of any one of claims 167-171, further comprising, prior to culturing said at least a portion of said PBMCs:
isolating CD14⁺ monocytes and CD3⁺ T cells from said population of PBMCs to yield said first portion of said PBMCs and said second portion of said PBMCs.

173. The method of any one of claims 167-172, wherein said increase in the concentration of the at least on cytokine in the first conditioned supernatant relative to the concentration of the at least one cytokine in the second conditioned supernatant is at least an increase of at least three times the concentration of the at least one cytokine in the second conditioned supernatant.

174. The method of any one of claims 167-173, wherein said monocyte checkpoint inhibitor is an anti-CD200L or anti-CD200 antibody or fragment thereof.

175. The method of any one of claims 167-174, further comprising, if said subject is in an inflammatory state and said at least one cytokine is TNF-α:
administering to said subject an anti-TNF-α therapy.

176. The method of any one of claims 167-174, further comprising, if said subject is in an inflammatory state and said at least one cytokine is IL-β:
administering to said subject an anti-IL-β therapy, either alone or in combination with an anti-TNF-α therapy.

177. The method of any one of claims 167-174, further comprising, if said subject is in an inflammatory state and said at least one cytokine is IL-6:
administering to said subject an anti-IL-6 therapy, either alone or in combination with an anti-TNF-α therapy.

178. The method of any one of claims 167-177, further comprising, if said subject is in an inflammatory state:
administering to said subject a composition comprising natural T cells (nT_{REGS}), manufactured regulatory T cells (iT_{REGS}), or a combination thereof.

179. The method of claim 178, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

180. The method of any one of claims 167-179, wherein said monocyte checkpoint inhibitor is present in said first culture medium at a concentration of 0.01 to 10 μg/mL.

181. The method of any one of claims 167-179, wherein said monocyte checkpoint inhibitor is present in said first culture medium at a concentration of 10 μg/mL.

182. A method for determining the inflammatory state of a subject suffering from amyotrophic lateral sclerosis (ALS), comprising:
isolating RNA or DNA from at least a portion of a sample comprising T cells from a subject suffering from ALS or isolating CD4⁺ and CD8⁺ T cell subsets from at least a portion of said sample to yield a nucleic acid sample;
quantifying the TCR repertoire diversity in the nucleic acid sample;
comparing said TCR repertoire diversity in the nucleic acid sample to a control sample or to a standard value,
wherein an increase in TCR repertoire diversity as indicated by an increase in the clonality index relative to the control sample or standard value is indicative that the subject is in an inflammatory state.

183. The method of claim 182, further comprising, if said subject is in an inflammatory state:
administering to said subject a composition comprising natural T cells (nT_{REGS}), manufactured regulatory T cells (iT_{REGS}), or a combination thereof.

184. The method of claim 183, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

185. The method of any one of claims 182-184, wherein said increase is at least 10%.

186. The method of any one of claims 182-184, wherein said increase is at least 20%.

187. The method of any one of claims 182-184, wherein said increase is at least 30%.

188. The method of any one of claims 182-184, wherein said increase is at least 40%.

189. The method of any one of claims 182-184, wherein said increase is at least 50%.

190. The method of any one of claims 182-184, wherein said increase is at least 100%.

191. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS comprising monocytes in a culture medium comprising IL-4 and GM-CSF to yield antigen-presenting cells (APCs);
culturing at least a portion of said population of PBMCs comprising T cells from said subject with said APCs loaded with motor neuron cell debris derived from a motor neuron cell line;
culturing control T cells with said APCs loaded with overlapping peptides that comprise superoxide dismutase (SOD)-1 and TAR-DNA binding protein (TDP)-43;
testing the T cells from said subject and the control T cells for reactivity to motor neuron cell line as measured by cytokine secretion in culture supernatants,
wherein an at least a three-fold increase in secretion of any one of the following cytokines relative to control T cells, IFN-gamma, GM-CSF, or TNF-alpha indicates that said subject is in an inflammatory state.
wherein increased reactivity of the T cells from said subject relative to the control T cells is indicative of an inflammatory state.

192. The method of claim 191, further comprising:
harvesting said sample comprising said population of PBMCs from said subject.

193. The method of any one of claims 191-192, wherein said increase is at least a three-fold increase in cytokine secretion.

194. A method for determining the inflammatory state of a subject suffering from amyotrophic lateral sclerosis (ALS), comprising:
measuring by Western blot the concentration of IRF3 and IRF7 in peripheral blood mononuclear cells (PBMCs) from a sample comprising a population of PBMCs from a subject suffering from ALS;
wherein an increase in the concentration of IRF3 or IRF7 as determined by Western Blot relative to a control sample or a standard value is indicative that said subject is in an inflammatory state.

195. A method for determining the inflammatory state of a subject suffering from amyotrophic lateral sclerosis (ALS), comprising:
measuring by RT-PCR the concentration of IRF3 and IRF7 in peripheral blood mononuclear cells (PBMCs) from a sample comprising a population of PBMCs from a subject suffering from ALS;
wherein an increase in the concentration of IRF3 or IRF7 as determined by RT-PCR relative to a control sample or a standard value is indicative that said subject is in an inflammatory state.

196. The method of any one of claims 194-195, further comprising:
harvesting said sample comprising said population of PBMCs from said subject.

197. The method of any one of claims 194-196, further comprising:
isolating CD3⁺ T cells or CD14⁺ monocytes from said sample to yield said PBMCs.

198. A method for determining the inflammatory state of a subject suffering from amyotrophic lateral sclerosis (ALS), comprising:
isolating proteins from peripheral blood mononuclear cells (PBMCs) from a sample comprising a population of PBMCs from a subject suffering from ALS;
measuring the expression of TRAF6 and at least one of TRAF2, TRAF3 and TRAF5 by Western blot or RT-PCR;
comparing said expression of TRAF6 to said at least one of TRAF2, TRAF3 and TRAF5,
wherein higher expression of TRAF6 relative to said at least one of TRAF2, TRAF3 and TRAF5 is indicative that said subject is in an inflammatory state.

199. The method of claim 198, further comprising:
harvesting said sample comprising said population of PBMCs from said subject.

200. The method of any one of claims 198-199, further comprising:
isolating CD3⁺ T cells or CD14⁺ monocytes from said sample to yield said PBMCs.

201. The method of any one of claims 198-200, wherein an at least three-fold increase in expression of TRAF6 relative to TRAF2, TRAF3 and TRAF6 is indicative that said subject is in an inflammatory state.

202. The method of any one of claims 198-201, further comprising, if said subject is in an inflammatory state:
administering to said subject a targeted pharmacologic inhibitor of TRAF6.

203. The method of any one of claims 198-202, further comprising, if said subject is in an inflammatory state:
administering to said subject a composition comprising natural T cells (nT_{REGS}), manufactured regulatory T cells (iT_{REGS}), or a combination thereof.

204. The method of claim 203, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

205. A method for determining the inflammatory state of a subject suffering from amyotrophic lateral sclerosis (ALS), comprising:
culturing at least a portion of peripheral blood mononuclear cells (PBMCs) from a sample comprising a population of PBMCs from a subject suffering from ALS comprising CD14⁺ monocytes and CD3⁺ T cells in a culture medium and an agonist or antagonist of the adenosine A₁, A_{2A}, A₂B or A₃ receptors for a period of time to yield cultured PBMCs;
collecting at least a portion of said cultured PBMCs;
treating at least a portion of said cultured PBMCs to yield a cell lysate;
measuring cyclic AMP (cAMP) in said cell lysate;
comparing said concentration of cyclic AMP to a concentration of cAMP in a control sample or a standard value,
wherein a reduction of the cAMP concentration in said cell lysate relative to said control sample or a standard value is indicative that said subject is in an inflammatory state.

206. The method of claim 205, further comprising:
harvesting said sample comprising said population of PBMCs from said subject.

207. The method of any one of claims 205-206, wherein said period of time is from about 0 hours to about 48 hours.

208. The method of claim 207, wherein said period of time is from about 0 hours to 2 hours.

209. The method of any one of claims 205-208, wherein said agonist or antagonist of the adenosine A₁, A_{2A}, A₂B or A₃ receptors is present said culture medium at a concentration between 0.01 to 10 μM.

210. The method of any one of claims 205-208, wherein said agonist or antagonist of the adenosine A₁, A_{2A}, A₂B or A₃ receptors is present said culture medium at a concentration of 1 μM.

211. The method of any one of claims 205-210, further comprising, prior to culturing said at least a portion of said PBMCs:
isolating CD14⁺ monocytes and CD3⁺ T cells from said population of PBMCs to yield said at least a portion of said PBMCs.

212. The method of claim 211, wherein said at least portion of said PBMCs comprises CD14⁺ monocytes without CD3⁺ T cells.

213. The method of claim 211, wherein said at least a portion of said PBMCs comprises CD3⁺ T cells without CD14⁺ monocytes.

214. The method of any one of claims 205-213, wherein said culture medium is supplemented serum, wherein said serum is human serum at a concentration of 5%.

215. The method of any one of claims 205-214, further comprising, if said subject is in an inflammatory state:
administering to said subject an anti-TNF-α therapy.

216. The method of any one of claims 205-214, further comprising, if said subject is in an inflammatory state:
administering to said subject an anti-IL-β therapy, either alone or in combination with an anti-TNF-α therapy.

217. The method of any one of claims 205-214, further comprising, if said subject is in an inflammatory state:
administering to said subject an anti-IL-6 therapy, either alone or in combination with an anti-TNF-α therapy.

218. The method of any one of claims 205-217, further comprising, if said subject is in an inflammatory state:
administering to said subject a composition comprising natural T cells (nT_{REGS}), manufactured regulatory T cells (iT_{REGS}), or a combination thereof.

219. The method of claim 218, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

219. A method for determining the inflammatory state of a subject suffering from amyotrophic lateral sclerosis (ALS), comprising:
culturing a first portion of peripheral blood mononuclear cells (PBMCs) from a sample comprising a population of PBMCs from a subject suffering from ALS comprising CD14⁺ monocytes and CD3⁺ T cells in a first culture medium and an agonist or antagonist of the adenosine A₁, A_{2A}, A₂B or A₃ receptors at a concentration of about 0.01 to 10 μM for a period of time to yield first cultured PBMCs;
culturing a second portion of said PBMCs from said sample comprising CD14⁺ monocytes and CD3⁺ T cells in a second culture medium for said period of time to yield second cultured PBMCs;
collecting said first cultured PBMCs and said second cultured PBMCs;
treating said first cultured PBMCs and said second cultured PBMCs to yield a first cell lysate and a second cell lysate, respectively;
measuring a concentration of cyclic AMP in said first cell lysate and a concentration of cyclic AMP in said second cell lysate;
comparing said concentration of cyclic AMP in said first cell lysate and said concentration of cyclic AMP in said second cell lysate,
wherein a reduction in the concentration of cyclic AMP in said first cell lysate relative to said second cell lysate is indicative that said subject is in an inflammatory state.

220. The method of claim 219, further comprising:
harvesting said sample comprising said population of PBMCs from said subject.

221. The method of any one of claims 219-220, wherein said first culture medium and said second culture medium are supplemented with 5% human serum.

222. The method of any one of claims 219-221, wherein said period of time is from about 0 hours to about 48 hours.

223. The method of any one of claims 219-221, wherein said period of time is from about 0 to about 2 hours.

224. The method of any one of claims 219-223, further comprising, prior to culturing said at least a portion of said PBMCs:
isolating CD14⁺ monocytes and CD3⁺ T cells from said population of PBMCs to yield said first portion of said PBMCs and said second portion of said PBMCs.

225. The method of any one of claims 219-224, wherein said reduction in said concentration of cyclic AMP in said first cell lysate relative to said second cell lysate is at least 25%.

226. The method of any one of claims 219-225, further comprising, if said subject is in an inflammatory state and said at least one cytokine is TNF-α:
administering to said subject an anti-TNF-α therapy.

227. The method of any one of claims 219-225, further comprising, if said subject is in an inflammatory state and said at least one cytokine is IL-β:
administering to said subject an anti-IL-β therapy, either alone or in combination with an anti-TNF-α therapy.

228. The method of any one of claims 219-225, further comprising, if said subject is in an inflammatory state and said at least one cytokine is IL-6:
administering to said subject an anti-IL-6 therapy, either alone or in combination with an anti-TNF-α therapy.

229. The method of any one of claims 219-228, further comprising, if said subject is in an inflammatory state:
administering to said subject a composition comprising natural T cells (nT_{REGS}), manufactured regulatory T cells (iT_{REGS}), or a combination thereof.

230. The method of claim 229, further comprising, prior to administering to said subject the composition comprising manufactured T_{REG} cells:
subjecting said subject to an immune depletion regimen to reduce at least a portion of CD4⁺ Th1 and CD8⁺ Tc1 cells in said subject.

231. The method of claim 182, further comprising:
administering to said subject a therapeutically effective amount of an anti-TNF-α therapy, an anti-IL-6 therapy, or an anti-IL-β therapy.

232. A method comprising the methods of claims 52, 120 and 182.

233. The method of any of the preceding claims, wherein the subject is receiving a therapy for ALS, and wherein if the subject is in an inflammatory state, the method further comprises adjusting said therapy.

234. The method of claim 233, wherein said adjustment is to increase the dosage of the therapy.

235. The method of claim 233, wherein said adjustment is to add an additional therapy selected from an anti-TNF-α therapy, an anti-IL-6 therapy, an anti-IL-1β therapy or administration of nTREGS, iTREGS or a combination of both.
